SEQUENCE LISTING (1 sequence)

SEQ ID NO: 1 - protein, 68 aa, Homo sapiens [sequence omitted]

(19) United States
(12) Patent Application Publication
(10) Pub. No.: US 2024/0115678 A1
(43) Pub. Date: Apr. 11, 2024

(54) PERSONALIZED MULTIDISCIPLINARY CANCER THERAPY

(71) Applicant: James VELTMEYER, La Jolla, CA (US)

(72) Inventor: James VELTMEYER, La Jolla, CA (US)

(21) Appl. No.: 18/453,237

(22) Filed: Aug. 21, 2023

Related U.S. Application Data
(60) Provisional application No. 63/399,701, filed on Aug. 21, 2022.

(51) Int. Cl. A61K 39/00; A61K 31/05; A61K 31/09; A61K 31/12; A61K 31/122; A61K 31/192; A61K 31/198; A61K 31/352; A61K 31/357; A61K 31/365; A61K 31/375; A61K 31/385; A61K 31/4418; A61K 31/4745; A61K 31/4748; A61K 31/506; A61K 31/519; A61K 31/53; A61K 33/04; A61K 33/30; A61K 35/60; A61K 36/82; A61K 39/395

(52) U.S. Cl. CPC A61K 39/0011; A61K 31/05; A61K 31/09; A61K 31/12; A61K 31/122; A61K 31/192; A61K 31/198; A61K 31/352; A61K 31/357; A61K 31/365; A61K 31/375; A61K 31/385; A61K 31/4418; A61K 31/4745; A61K 31/4748; A61K 31/506; A61K 31/519; A61K 31/53; A61K 33/04; A61K 33/30; A61K 35/60; A61K 36/82; A61K 39/3955; A61K 2039/55516

(57) ABSTRACT

Disclosed are assessment means, treatment protocols and compositions of matter for the personalized therapy of neoplastic malignancy. In one embodiment a patient is assessed for various body and cancer parameters and a multistep approach is undertaken which addresses: a) hormonal abnormalities; b) tumor genetic composition; c) immunological status of the tumor; d) nutrient deficiencies; e) oxidative stress associated with the tumor; and f) tumor acidity. Subsequent and/or concurrent with addressing abnormalities various therapeutic interventions are utilized including immunotherapy, chemotherapy, radiotherapy and metabolic therapy. Immunization means are also provided to prevent the possibility of tumor relapse.

### CROSS-REFERENCE TO RELATED APPLICATIONS

This application claims priority to U.S. Provisional Application No. 63/399,701, titled “Personalized Multidisciplinary Cancer Therapy”, filed on Aug. 21, 2022, which is hereby incorporated by reference in its entirety.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 19, 2023, is named “JV_PMCT_NP1_SL.xml” and is 2 kilobytes in size. The sequence listing contained in this .XML file is part of the specification and is hereby incorporated by reference herein in its entirety.

### FIELD OF THE INVENTION

The teachings herein relate to methods of treating cancer involving reducing tumor-associated immune suppression.

### SEQUENCE LISTINGS

SEQ ID NO: 1
EFDVILKAAGANKVAVIKAVRGATGLGLKEAKDLVESAPAALKEGVSKD
DAEALKKALEEAGAEVEVK.

### BACKGROUND

It is the belief of many that cancer immunotherapy was born at the turn of the 20^{th} Century with the work of William Coley, who was able to successfully induce tumor regression by administration of a mixture consisting of killed bacteria of species Streptococcus pyogenes and Serratia marcescens [1-8]. In more recent years, the FDA approval of the Provenge dendritic cell vaccine for prostate cancer (2010) [9], Ipilimumab (Yervoy) anti-CTLA4 antibody for treatment of melanoma (2011) [10], Pembrolizumab (Keytruda) anti-PD1 antibody for melanoma (2014) [11], and Nivolumab (Opdivo) for melanoma (2014) [12], ushered a new age of immunotherapy. Despite improved survival using these novel immune modulators, not all patients respond, with an average of 20% remissions being reported [13].

The current invention was developed to address multiple aspects of the immune system in order to augment possibility of increasing overall survival. Specifically, it is known from studies of immune modulators that recruitment of multiple arms of the immune system associates with increased efficacy. For example, it is known that natural killer cells play an important role in immune destruction of cancer [14-20]. A clinical trial demonstrated that patients who possess elevated levels of natural killer cell inhibitory proteins (soluble NKG2D ligands) demonstrated lower responses to checkpoint inhibitors [21]. Indeed this should not be surprising since studies show that NK cell infiltration of tumors induces upregulation of antigen presentation in an interferon gamma associated manner, which renders tumor cells sensitive to T cell killing [22]. Another example of the potency of combining immunotherapies is the example of Herceptin, in which approximately 1 out of 4 patients with the HER2neu antigen respond to treating. Interestingly it was found that lack of responsiveness correlates with inhibited NK cell activity [23-25]. Indeed, animal experiments demonstrate augmentation of Herceptin activity by stimulators of NK cells such as Poly (IC) and IL-12 [26, 27]. The current invention aims to integrate the main arms of the immune system so as to achieve a synergistic induction of anticancer immunity.

### SUMMARY

Preferred embodiments include methods of treating cancer comprising: a) examining a cancer patient for tumor and host associated abnormalities; b) addressing said abnormalities; c) providing one or more therapeutic interventions; and d) providing immunological support to avoid tumor recurrence.

Preferred methods include embodiments wherein said therapeutic capable of reducing tumor-associated immune suppression is an antioxidant.

Preferred methods include embodiments wherein said antioxidant is selected from a group comprising of: a) n-acetylcysteine; b) intravenous ascorbic acid; c) pterostilbene; d) vitamin k3; e) resveratrol; f) alpha lipoic acid; g) quercetin; h) kaempferol; i) myricetin; j) apigenin; k) luteolin; l) curcumin; and m) caffeic acid.

Preferred methods include embodiments wherein said therapeutic capable of reducing tumor-associated immune suppression is a phosphodiesterase (PDE)-5 inhibitor.

Preferred methods include embodiments wherein said PDE-5 inhibitor is selected from a group comprising of: a) Acetildenafi; b) Aildenafil; c) Avanafil; d) Benzamidenafil; e) Homosildenafil; f) Icariin; g) Lodenafil; h) Mirodenafil; i) Nitrosoprodenafil; j) Sildenafil; k) Sulfoaildenafil; l) Tadalafil; m) Udenafil; n) Vardenafil; and o) Zaprinast

Preferred methods include embodiments wherein said therapeutic capable of reducing tumor-associated immune suppression is nitroglycerin.

Preferred methods include embodiments wherein said therapeutic capable of reducing tumor-associated immune suppression is an agent capable of reducing VEGF.

Preferred methods include embodiments wherein said agent capable of reducing said VEGF is selected from a group comprising of: a) Avastin; b) Ciclopirox; c) penicillamine; d) tetrathiomolybdate; e) fish oil; f) selenium; g) green tea polyphenols; h) glycine; i) zinc; j) cirsimaritin; k) Eupafolin; l) Andrographolide; m) Procyanidin B2; n) Procyanidin B3; o) 6-O-angeloylenolin; p) Cyperenoic acid; q) Penduliflaworosin; r) Tylophorine; s) Ellagic acid; t) brucine; u) Punarnavine; v) Raddeanin A; w) Platycodin D; x) withanone; y) 4-Hydroxyphenylacetic acid; z) trans-ethyl p-methoxycinnamate; aa) Decursin; ab) decursinol angelate; and ac) Artesunate.

Preferred methods include embodiments wherein said therapeutic capable of reducing tumor-associated immune suppression is a checkpoint inhibitor.

Preferred methods include embodiments wherein said checkpoint inhibitor is an agent capable of blocking molecules selected from a group comprising of: a) PD-1; b) PD-L1; c) CTLA-4; d) LAG-3; e) TIGIT; f) KIR; g) indolamine 2,3 deoxygenase; h) NR2F6; i) TIM-3; j) ILT-3; and k) GITR.

Preferred methods include embodiments wherein said patient is immunized with a tumor antigen, wherein said tumor antigen possesses similarity to said tumor which said patient is afflicted by.

Preferred methods include embodiments wherein said tumor antigen is derived from a histologically similar tumor to which said patient is afflicted with.

Preferred methods include embodiments wherein said tumor antigen is derived by lysis of histologically similar tumors.

Preferred methods include embodiments wherein said tumor antigen is derived by mRNA extraction of histologically similar tumors.

Preferred methods include embodiments wherein said tumor antigen is derived by exosome extraction of histologically similar tumors.

Preferred methods include embodiments wherein said tumor antigen is a tumor associated protein.

Preferred methods include embodiments wherein said tumor associated protein is selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYPIB1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4)

Preferred methods include embodiments wherein a peptide or plurality of peptides derived from said antigens are used for immunization.

Preferred methods include embodiments wherein said peptides used for immunization are matched with HLA haplotype of said patient in need of therapy.

Preferred methods include embodiments wherein said peptides are altered peptide ligands.

Preferred methods include embodiments wherein said immunization with said tumor antigen is performed together with an adjuvant.

Preferred methods include embodiments wherein said adjuvant is a stimulator of antigen presentation.

Preferred methods include embodiments wherein said stimulator of antigen presentation is a toll like receptor (TLR).

Preferred methods include embodiments wherein said toll like receptor is TLR-2.

Preferred methods include embodiments wherein said TLR-2 is activated by compounds selected from a group comprising of: a) Pam3cys4; b) Heat Killed Listeria monocytogenes (HKLM); and c) FSL-1.

Preferred methods include embodiments wherein said toll like receptor is TLR-3.

Preferred methods include embodiments wherein said TLR-3 is activated by Poly IC.

Preferred methods include embodiments wherein said TLR-3 is activated by double stranded RNA.

Preferred methods include embodiments wherein said double stranded RNA is of mammalian origin.

Preferred methods include embodiments wherein said double stranded RNA is of prokaryotic origin.

Preferred methods include embodiments wherein said double stranded RNA is derived from leukocyte extract.

Preferred methods include embodiments wherein said leukocyte extract is a heterogeneous composition derived from freeze-thawing of leukocytes, followed by dialysis for compounds less than 15 kDa.

Preferred methods include embodiments wherein said toll like receptor is TLR-4.

Preferred methods include embodiments wherein said TLR-4 is activated by lipopolysaccharide.

Preferred methods include embodiments wherein said TLR-4 is activated by peptide possessing at least 80 percent homology to the sequence (SEQ ID NO: 1) EFDVILKAAGANKVAVIKAVRGATGLGLKEAKDLVESAPAALKEGVSKDDAEALKKAL EEAGAEVEVK.

Preferred methods include embodiments wherein said TLR-4 is activated by HMGB-1.

Preferred methods include embodiments wherein said TLR-4 is activated by a peptide derived from HMGB-1.

Preferred methods include embodiments wherein said HMGB-1 peptide is hp91.

Preferred methods include embodiments wherein said toll like receptor is TLR-5.

Preferred methods include embodiments wherein said TLR-5 is activated by flagellin.

Preferred methods include embodiments wherein said toll like receptor is TLR-7.

Preferred methods include embodiments wherein said TLR-7 is activated by imiquimod.

Preferred methods include embodiments wherein said toll like receptor is TLR-8.

Preferred methods include embodiments wherein said TLR-8 is activated by resmiqiumod.

Preferred methods include embodiments wherein said toll like receptor is TLR-9

Preferred methods include embodiments wherein said TLR-9 is activated by CpG DNA.

Preferred methods include embodiments wherein said stimulator of antigen presentation is an agent capable of upregulating expression of costimulatory molecules on antigen presenting cells.

Preferred methods include embodiments wherein said costimulatory molecules are selected from a group comprising of: a) CD40; b) CD80; and c) CD86.

Preferred methods include embodiments wherein said agent capable of upregulating expression of costimulatory molecules is an activator of NF-kappa B.

Preferred methods include embodiments wherein said activator of NF-kappa B is an inhibitor of i-kappa B.

Preferred methods include embodiments wherein said agent capable of inducing upregulation of costimulatory molecules is an activator of the JAK-STAT pathway.

Preferred methods include embodiments, wherein said agent capable of upregulating activity of the JAK-STAT pathway is interferon gamma.

Preferred methods include embodiments wherein said activator of NF-kappa B is an activator of a Pathogen Associated Molecular Pattern (PAMP) receptor.

Preferred methods include embodiments wherein said PAMP receptor is selected from a group comprising of: a) MDA5; b) RIG-1; and c) NOD.

Preferred methods include embodiments wherein said agent capable of activating antigen presentation locally is a dendritic cell.

Preferred methods include embodiments wherein said dendritic cell is activated with a TLR agonist.

Preferred methods include embodiments wherein said dendritic cell is activated with a PAMP agonist.

Preferred methods include embodiments wherein said dendritic cell is generated from patient monocytes.

Preferred methods include embodiments wherein said dendritic cell is autologous to the patient in need of treatment.

Preferred methods include embodiments wherein said dendritic cell is allogeneic to the patient in need of treatment.

Preferred methods include embodiments wherein said dendritic cell is activated in vivo by administration of GM-CSF.

Preferred methods include embodiments wherein said dendritic cell is activated in vivo by administration of FLT-3L.

Preferred methods include embodiments wherein said means of induction of localized tumor cell death is achieved by administration of localized radiation therapy.

Preferred methods include embodiments wherein said means of induction of localized tumor cell death is achieved by cryoablation.

Preferred methods include embodiments wherein said means of induction of localized tumor cell death is achieved by localized administration of hyperthermia.

Preferred methods include embodiments wherein said means of induction of localized tumor cell death is achieved by localized administration of chemotherapy.

Preferred methods include embodiments wherein said chemotherapy is selected from a group comprising of: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; bisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflornithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; hydroxyurea; idarubicin hydrochloride; ifosfamide; ilmofosine; interleukin II (including recombinant interleukin II, or rIL2), interferon alfa-2a; interferon alfa-2b; interferon alfa-n1; interferon alfa-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Preferred methods include embodiments wherein prior to intervention a state of lymphopenia is induced in said patient in need of treatment.

Preferred methods include embodiments wherein said lymphopenia is sufficient to induce homeostatic expansion of lymphocytes in said patient.

Preferred methods include embodiments wherein said lymphopenia is sufficient to induce homeostatic proliferation of lymphocytes residing in patient in need of treatment.

Preferred methods include embodiments wherein said homeostatic expansion allows for an over 50% reduction in need of said lymphocytes for costimulatory signals.

Preferred methods include embodiments wherein said lymphopenia is achieved by irradiation.

Preferred methods include embodiments wherein said irradiation is total lymphoid irradiation.

Preferred methods include embodiments wherein said lymphopenia is induced by administration of cyclophosphamide.

Preferred methods include embodiments wherein increased propensity of lymphocytes for activation is induced by treatment with a lymphocyte mitogen.

Preferred methods include embodiments wherein said lymphocyte mitogen comprises of interleukin-2 treatment.

Preferred methods include embodiments wherein said lymphocyte mitogen comprises of interleukin-7 treatment.

Preferred methods include embodiments wherein said lymphocyte mitogen comprises of interleukin-15 treatment.

Preferred methods include embodiments wherein said tumor is a brain tumor.

Preferred methods include embodiments wherein said brain tumor is selected from a group comprising of: a) a glioblastoma; b) a glioblastoma multiforme; c) an oligodendroglioma; d) a primitive neuroectodermal tumor; e) an astrocytoma; f) an ependymoma; g) an oligodendroglioma; h) a medulloblastoma; i) a meningioma; j) a pituitary carcinoma; k) a neuroblastoma; or l) a craniopharyngioma.

Preferred methods include embodiments wherein said abnormality is abnormal hormonal status.

Preferred methods include embodiments wherein is abnormal hormonal status is associated with enhanced tumor growth.

Preferred methods include embodiments wherein is abnormal hormonal status is associated with enhanced tumor angiogenesis.

Preferred methods include embodiments wherein is abnormal hormonal status is associated with enhanced tumor metastasis.

Preferred methods include embodiments wherein is abnormal hormonal status is associated with enhanced tumor immune suppression.

Preferred methods include embodiments wherein said patient is supplemented with growth hormone at a concentration and quantity sufficient to augment T cell responses.

Preferred methods include embodiments wherein said patient is supplemented with growth hormone at a concentration and quantity sufficient to augment NK cell responses.

Preferred methods include embodiments wherein said tumor abnormality is the tumor genetic composition.

Preferred methods include embodiments wherein said tumor genetic composition is the microsatellite status of the tumor.

Preferred methods include embodiments wherein said tumor genetic composition is utilized to generate tumor-specific vaccines.

### DETAILED DESCRIPTION OF THE INVENTION

In the current invention we teach the prior sensitization of tumors by systemic immunization, followed by induction of immunogenic cell death, followed by augmentation of tumor specific immune responses.

Various embodiments of the present invention provide a method of treating, reducing the severity of and/or slowing the progression of a tumor in a subject. The method may consist of or may comprise: providing an immunization means which activates effector cells to expand in vivo, followed by administration of a therapeutically effective amount of antigen presenting cells, such as dendritic cells into the local tumor microenvironment, followed by induction of immunogenic tumor cell death, followed by administration of agents, or vaccines capable of eliciting immunosurveillance to prevent tumor relapse, as well as to induce an abscopal effect. In various embodiments, the immune cell is primed against a tumor cell lysate, tumor cell antigen, tumor cell cytokine, and/or stem cell lysate.

Unless defined differently, all technical and scientific terms used herein have the same meanings as commonly understood by one of skill in the art to which the disclosed invention belongs. In particular, the following terms and phrases have the following meaning.

“Adjuvant” refers to a substance that is capable of enhancing, accelerating, or prolonging an immune response when given with a vaccine immunogen.

“Agonist” refers to is a substance which promotes (induces, causes, enhances or increases) the activity of another molecule or a receptor. The term agonist encompasses substances which bind receptor (e.g., an antibody, a homolog of a natural ligand from another species) and substances which promote receptor function without binding thereto (e.g., by activating an associated protein).

“Antagonist” or “inhibitor” refers to a substance that partially or fully blocks, inhibits, or neutralizes a biological activity of another molecule or receptor.

“Co-administration” refers to administration of two or more agents to the same subject during a treatment period. The two or more agents may be encompassed in a single formulation and thus be administered simultaneously. Alternatively, the two or more agents may be in separate physical formulations and administered separately, either sequentially or simultaneously to the subject. The term “administered simultaneously” or “simultaneous administration” means that the administration of the first agent and that of a second agent overlap in time with each other, while the term “administered sequentially” or “sequential administration” means that the administration of the first agent and that of a second agent does not overlap in time with each other.

“Immune response” refers to any detectable response to a particular substance (such as an antigen or immunogen) by the immune system of a host vertebrate animal, including, but not limited to, innate immune responses (e.g., activation of Toll receptor signaling cascade), cell-mediated immune responses (e.g., responses mediated by T cells, such as antigen-specific T cells, and non-specific cells of the immune system), and humoral immune responses (e.g., responses mediated by B cells, such as generation and secretion of antibodies into the plasma, lymph, and/or tissue fluids). Examples of immune responses include an alteration (e.g., increase) in Toll-like receptor activation, lymphokine (e.g., cytokine (e.g., Th1, Th2 or Th17 type cytokines) or chemokine) expression or secretion, macrophage activation, dendritic cell activation, T cell (e.g., CD4+ or CD8+T cell) activation, NK cell activation, B cell activation (e.g., antibody generation and/or secretion), binding of an immunogen (e.g., antigen (e.g., immunogenic polypolypeptide)) to an MHC molecule, induction of a cytotoxic T lymphocyte (“CTL”) response, induction of a B cell response (e.g., antibody production), and, expansion (e.g., growth of a population of cells) of cells of the immune system (e.g., T cells and B cells), and increased processing and presentation of antigen by antigen presenting cells. The term “immune response” also encompasses any detectable response to a particular substance (such as an antigen or immunogen) by one or more components of the immune system of a vertebrate animal in vitro.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasioa). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example,/pindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.” The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha..beta.T cell expressing TCR .alpha. and .beta. chains, and a .gamma..delta.T cell expressing TCR .gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used. The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain. The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered. The terms “individual”, “host”, “subject”, and “patient” are used interchangeably herein, and refer to a mammal, including, but not limited to, primates, for example, human beings, as well as rodents, such as mice and rats, and other laboratory animals.

The term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside.

The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

In one embodiment of the invention, immunization to tumors of the same type the patient is suffering from is provided prior to cytotoxic, or immunogenic cell death induction of the tumor. Immunization of the patient may be performed using known means in the art, using suitable adjuvants. Assessment of immunity is performed by quantifying reactivity of T cells or B cells in response to protein antigens or derivatives thereof, derivatives including peptide antigens or other antigenic epitopes. Responses may be assessed in terms of proliferative responses, cytokine release, antibody responses, or generation of cytotoxic T cells. Methods of assessing said responses are well known in the art. In a preferred embodiment, antibody responses are assessed to a panel of tumor associated proteins subsequent to immunization of patient. Antibody responses are utilized to guide which peptides will be utilized for prior immunization. For example, if a patient is immunized with tumor antigen on a weekly basis, the subsequent assessment of antibody responses is performed at approximately 1-3 months after initiation of immunization. Protocols for immunization include weekly, biweekly, or monthly. Assessment of antibody responses is performed utilizing standard enzyme linked immunosorbent (ELISA) assay. Assessment of antibodies is performed, in one embodiment of the invention, against proteins associated with tumor.

In one embodiment of the invention, immunity to a polyvalent tumor vaccine is induced utilizing a vaccine such as CanVaxin [28, 29], or other polyvalent vaccine mixtures. Numerous tumor antigens can be utilized to amplify the immune response selectively, these can be chosen from known groups of tumor antigens such as ERG, WT1, ALS, BCR-ABL, Ras-mutant, MUC1, ETV6-AML, LMP2, p53 non-mutant, MYC-N, surviving, androgen receptor, RhoC, cyclin B1, EGFRvIII, EphA2, B cell or T cell idiotype, ML-IAP, BORIS, hTERT, PLAC1, HPV E6, HPV E7, OY-TES1, Her2/neu, PAX3, NY-BR-1, p53 mutant, MAGE A3, EpCAM, polysialic Acid, AFP, PAX5, NY-ESO1, sperm protein 17, GD3, Fucosyl GM1, mesothelin, PSMA, GD2, MAGE A1, sLe(x), HMWMAA, CYPIB1, sperm fibrous sheath protein, B7H3, TRP-2, AKAP-4, XAGE 1, CEA, Tn, GloboH, SSX2, RGS5, SART3, gp100, MelanA/MART1, Tyrosinase, GM3 ganglioside, Proteinase 3 (PR1), Page4, STn, Carbonic anhydrase IX, PSCA, Legumain, MAD-CT-1 (protamin2), PSA, Tie 2, MAD-CT2, PAP, PDGFR-beta, NA17, VEGFR2, FAP, LCK, Fos-related antigen, LCK, FAP.

Combination of polyvalent vaccines with other cellular therapies as the initial poly-immunogenic composition is envisioned within the context of the invention. In one embodiment cellular lysates of tumor cells, or tumor stem cells are loaded into dendritic cells. In one embodiment the invention provides a means of generating a population of cells with tumoricidal ability that are polyvalently reactive, to which focus is added by subsequent peptide specific vaccination. The generation of cytotoxic lymphocytes may be performed, in one embodiment by extracted 50 ml of peripheral blood from a cancer patient and peripheral blood monoclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generates a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor antigen. Pulsing may be performed by incubation of lysates with dendritic cells, or may be generated by fusion of immature dendritic cells with tumor cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-7). During the course of generating DC for clinical purposes, microbiologic monitoring tests are performed at the beginning of the culture, on the fifth day and at the time of cell freezing for further use or prior to release of the dendritic cells. Administration of tumor pulsed dendritic cells is utilized as a polyvalent vaccine, whereas subsequent to administration antibody or t cell responses are assessed for induction of antigen specificity, peptides corresponding to immune response stimulated are used for further immunization to focus the immune response.

In some embodiments, culture of the immune effectors cells is performed after extracting from a patient that has been immunized with a polyvalent antigenic preparation. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumors and subsequently injected into the patient in need of treatment. Expansion with specific antigens includes coculture with proteins selected from a group comprising of: a) ROBO; b) VEGF-R2; c) FGF-R; d) CD105; e) TEM-1; and f) survivin. Other tumor specific or semi-specific antigens are known in the art that may be used.

Within the context of the invention, teachings are provided to amplify an antigen specific immune response following immunization with a polyvalent vaccine, in which the antigenic epitopes are used for immunization together with adjuvants such as toll like receptors (TLRs). These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T.sub.H1-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments of the invention, specific antigens are immunized following polyvalent immunization, said specific antigens administered in the form of DNA vaccines. Numerous publications have reported animal and clinical efficacy of DNA vaccines which are incorporated by reference [30-32]. In addition to direct DNA injection techniques, DNA vaccines can be administered by electroporation [33]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N-[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N—(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [34]. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed [35]. PMED is a needle-free method of administering vaccines to animals or humans. The PMED system involves the precipitation of DNA onto microscopic gold particles that are then propelled by helium gas into the epidermis [36]. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation [37]. Electroporation uses controlled electrical pulses to create temporary pores in the cell membrane, which facilitates cellular uptake of the nucleic acid vaccine injected into the muscle [38-41]. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

Subsequent to augmentation of lymphocyte numbers specific for killing of the tumor, modification of the tumor microenvironment may be performed. In one embodiment, macrophage modulators are used.

Macrophages are key components of the innate immune system which play a principal role in the regulation of inflammation as well as physiological processes such as tissue remodeling [42, 43]. The diverse role of macrophages can be seen in conditions ranging from wound healing [44-47], to myocardial infarction [48-54], to renal failure [55-58] and liver failure [59].

Differentiated macrophages and their precursors are versatile cells that can adapt to microenvironmental signals by altering their phenotype and function [60]. Although they have been studied for many years, it has only recently been shown that these cells comprise distinct sub-populations, known as classical M1 and alternative M2 [61]. Mirroring the nomenclature of Th1 cells, M1 macrophages are described as the pro-inflammatory sub-type of macrophages induced by IFN-.gamma. and LPS. They produce effector molecules (e.g., reactive oxygen species) and pro-inflammatory cytokines (e.g., IL-12, TNF-.alpha. and IL-6) and they trigger Th1 polarized responses [62].

Macrophages can play a tumor inhibitory, as well as a tumor stimulatory role. Initial studies supported the role of macrophages in mediating antibody dependent cellular cytotoxicity in tumors [63-70], and thus being associated with potentiation of antitumor immune responses. Macrophages also possess the ability to directly recognize tumors by virtue of tumor expressed “eat-me” signals, which include the stress associated protein calreticulin [71, 72], which binds to the low-density lipoprotein receptor-related protein (LRP) on macrophages to induce phagocytosis [73]. Tumors protect themselves by expression of CD47, which binds to macrophage SIRP-1 and transduces an inhibitory signal [74]. Blockade of CD47 using antibodies results in remission of cancers mediated by macrophage activation [75-79]. Thus on the one hand, macrophages play an important role in induction of antitumor immunity. This can also be exemplified by some studies, involving administration of GM-CSF in order to augment macrophage numbers and activity in cancer patients [80-83].

Unfortunately, there is also evidence that macrophages support tumor growth. Studies in the osteopetrotic mice strain, which lacks mature macrophages, demonstrate that tumors actually grow slower in animals deficient in macrophages [84]. Several other animal models have elegantly demonstrated that macrophages contribute to tumor growth, in part through stimulating on the angiogenic switch [85-87]. Numerous tumor biopsy studies have shown that there is a negative correlation between macrophage infiltration into tumors and patient survival [88-92].

The duality of macrophages in growth of tumors may be seen in studies of “inverse hormesis” in which low concentrations of antibodies targeting the tumor specific marker sialic acid N-glycolyl-neuraminic acid (Neu5Gc) actually leads to enhanced tumor growth in a macrophage dependent manner [93].

The importance of macrophages in clinical implementation of cancer therapeutics can be seen from results of a double blind clinical trials in metastatic colorectal cancer patients where cetuximab (anti-epidermal growth factor receptor (EGFR) monoclonal antibody (mAb)) was added to a protocol comprising of bevacizumab and chemotherapy. The addition of cetuximab actually resulted in decreased survival. In a study examining whether monocyte conversion to M2 angiogenic macrophages was responsible, investigators observed that CD163-positive M2 macrophages where found in high concentrations intratumorally in patients with colorectal carcinomas. These M2 cells expressed abundant levels of Fc-gamma receptors (FcTR) and PD-L1. Additionally, consistent with the M2 phenotype the cells generated large amounts of the immunosuppressive molecule IL-10 and the angiogenic mediator VEGF. When M2 cells were cultured with EGFR-positive tumor cells loaded with low concentrations of cetuximab, further augmentation of IL-10 and VEGF production was observed. These data suggest that under certain contexts, tumors manipulate macrophages to take on the M2 phenotype, and this subsequently leads to enhanced tumor progressing factors when tumor cells are bound by antibodies [94].

Manipulation of macrophages to inhibit M2 and shift to M1 phenotype may be performed using a variety of means. One theme that seems unifying is the ability of toll like receptor (TLR) agonists to influence this. In addition to cytokine differences, macrophages capable of killing tumor cells are usually known to express low levels of the inhibitory Fc gamma receptor IIb, whereas tumor promoting macrophages have high levels of this receptor [95]. Furthermore, tumor associated cytokines such as IL-4 and IL-10 are known to induce upregulation of the Fc gamma receptor IIB [96-99].

In one study, the effect of the TLR7/8 agonist R-848 was assessed on monocytes derived from human peripheral blood. It was found that 12 hour exposure of R-848 increased FcgammaR-mediated cytokine production and antibody-dependent cellular cytotoxicity by monocytes. Furthermore, upregulation of the ADCC associated receptors FcgammaRI, FcgammaRIIa, and the common gamma-subunit was observed. However treatment with R-848 led to profound downregulation of the inhibitory FcgammaRIIb molecule [100]. These data support ability to modify therapeutic activity of macrophages by manipulation of TLR signaling pathways. Other TLRs have been found to suppress inhibitory receptors on macrophages. For example, in another study it was observed that exposing monocytes to TLR4 agonists leads to suppression of the FcTRIIb macrophage inhibitory protein by MARCH3 mediated ubiquitination [101].

In one embodiment administration of ImmunoMax is performed systemically, and/or locally, which is an injectable polysaccharide purified from potato sprouts and approved as pharmaceutical in the Russian Federation (registration P No. 001919/02-2002) and 5 other countries of Commonwealth of Independent States (formerly the USSR) and has been evaluated in a wide range of medical situations. In accordance with the formal “Instruction of Medical Use”, one medical indication for Immunomax® is the stimulation of immune defense during the treatment of different infectious diseases (http://www.gepon.ru/immax_intro.htm). Studies have shown that Immunomax® induces immune mediated killing of cancer cells in a TLR4 dependent manner [102]. In one embodiment of the invention, ImmunoMax is utilized to induce an M2 to M1 shift, thus reducing macrophage derived immune suppressants and augmenting production of immune stimulatory cytokines such as IL-12 and TNF-alpha [102]. In some embodiments of the invention, other agents may be used to modulate M2 to M1 transition of tumor associated macrophages including RRx-001 [103], the bee venom derived peptide melittin [104], CpG DNA [105, 106], metformin [107], Chinese medicine derivative puerarin [108], rhubarb derivative emodin [109], dietary supplement chlorogenic acid [110], propranolol [111], poly ICLC [112], BCG [113], Agaricus blazei Murill mushroom extract [114], endotoxin [115], olive skin derivative maslinic acid [116], intravenous immunoglobulin [117], phosphotidylserine targeting antibodies [118], dimethyl sulfoxide [119], surfactant protein A [120], Zoledronic acid [121], bacteriophages [122],

Prior to induction of immunogenic cell death, antigen presenting cells are administered within the current invention, one of the most potent antigen presenting cells is the dendritic cell.

Dendritic cells (DC) possess unique morphology similar to neuronal dendrites and were originally identified based on their ability to stimulate the adaptive immune system. Of importance to the field of tumor immunotherapy, dendritic cells appear to be the only cell in the body capable of activating naïve T cells [123]. The concept of dendritic cells instructing naïve T cells to differentiate into effector or memory cells is fundamental because it places the dendritic cell as the most powerful antigen presenting cell. This implies that for immunotherapeutic purposes dendritic cells do not necessarily need to be administered at high numbers in patients. One way in which dendritic cells have been described is as sentinels of the immune system that are patrolling the body in an immature state [124, 125]. Once DC are activated, by a stimulatory signal such as a Damage Associated Molecular Patterns (DAMPS) the DC then migrate into the draining lymph nodes through the afferent lymphatics. During the trafficking process, DC degrade ingested proteins into peptides that bind to both MHC class I molecules and MHC class II molecules. This allows the DC to: a) perform cross presentation in that they ingest exogenous antigens but present peptides in the MHC I pathway; and b) activate both CD8 (via MHC I) and CD4 (via MHC II). Interestingly, lipid antigens are processed via different pathways and are loaded onto non-classical MHC molecules of the CD1 family [126].

The possibility of utilizing DC to stimulate immunity was made into reality in animal studies that took advantage of the ability of immature DC to potently phagocytose various antigens. If the antigens possessed DAMPs, or if DAMPs were present in the environment, the DC would mature and present the antigens, resulting in stimulation of potent T cell immunity. Accordingly, in the initial studies, immature DC were incubated with various antigens, subsequent to which a maturation signal (replicating natural DAMPs) was applied and the DC were injected into animals. Thus DC were utilized as a type of “cellular adjuvant”. Indeed, it was discovered that the classical adjuvants such as Fruend's Adjuvant actually contained a high concentration of DAMPs, which resulted in the stimulation of local DC at vaccination site in vivo.

One of the first clinical applications of DC was prostate cancer. In an early reported, thirty three androgen resistant metastatic prostate cancer patients were treated with DC that were pulsed with peptides from a prostate specific antigen termed PMSA. Nine partial responders were identified based on NCPC criterial, plus 50% reduction of PSA. Four of the partial responders were also responders in the phase I study, with an average response duration of 225 days. Their combined average total response period was over 370 days. Five other responders in the secondary immunizations at the Phase II were nonresponders in the phase I study. Their average partial response period was 196 days. These data support the safety of follow-up infusion of DC that have been pulsed with tumor antigen derived peptide [127].

The same group published a subsequent paper on an additional 33 patients that had not received prior DC immunization in the Phase I. All subjects received six infusions of DC pulsed with PSM-P1 and -P2 at six week intervals without any treatment associated adverse events. Six partial and two complete responders were identified in the phase II study based on NPCP criteria, plus 50% reduction of prostate-specific antigen (PSA), or resolution in previously measurable lesions on ProstaScint scan [128]. The same group analyzed immune response in patients who had clinical remission or relapsed. A strong correlation was found between delayed type hypersensitivity response to the PSM-P1 and PSM-P2 and clinical response [129].

Another subsequent study utilized DC generated using GM-CSF and IL-4 but pulsed with PAP, another prostate antigen. Specifically, the PAP was delivered to the DC by means of generation of a PAP-GM-CSF fusion protein. Two intravenous infusions of the generated cells were performed one month apart in 12 patients with androgen resistant prostate cancer. The infusions were followed by three s.c. monthly doses of the fusion protein without cells. Treatment was well tolerated and circulating prostate-specific antigen levels dropped in three patients. Immune response to the fusion protein was observed, as well as to PAP [130]. In addition to prostate cancer, in which FDA approval has been granted for the Provenge drug, numerous trials have been conducted in a wide variety of cancers. All the trials demonstrated safety, without serious adverse effects of DC administration, as well as some degree of therapeutic efficacy. Trials have been conducted in melanoma [131-182], soft tissue sarcoma [183], thyroid [184-186], glioma [187-208], multiple myeloma, [209-217], lymphoma [218-220], leukemia [221-228], as well as liver [229-234], lung [235-248], ovarian [249-252], and pancreatic cancer [253-255].

Within the context of the invention, T cell activation is performed in vivo. In one embodiment, transfer factor is utilized. T cells are immune effectors against tumors, possessing ability to directly kill via CD8 cytotoxic cells [256-258], or indirectly killing tumors by activation of macrophages through interferon gamma production [259-261]. Additionally, T cells have been shown to convert protumor M2 macrophages to M1 [262]. The importance of T cells in cancer is illustrated by positive correlation between tumor infiltrating lymphocytes and patient survival [263-267]. In addition, positive correlations between responses to various immunotherapies has been made with tumor infiltrating lymphocyte density [268, 269]. Increased T cell activity is associated with reduction in T regulatory (Treg) cells. Studies show that agents that cause suppression of Treg cells correlates with improved tumor control. Agents that inhibit Treg cells include arsenic trioxide [270], cyclophosphamide [271-273], triptolide [272], gemcitabine [274], and artemether [275].

T cell modulator (TCM) is a pharmaceutical grade transfer factor, which activates T cells by reducing costimulatory requirements, thus potentially increasing infiltration of tumors by T cells. The concept of an immunologically acting “Transfer Factor” was originally identified by Henry Lawrence in a 1956 publication [276], in which he reported simultaneous transfer of delayed hypersensitivity to diphtheria toxin and to tuberculin in eight consecutive healthy volunteers who received extracts from washed leucocytes taken from the peripheral blood of tuberculin-positive, Schick-negative donors who were highly sensitive to purified diphtheria toxin and toxoid. The leucocyte extracts used for transfer contained no detectable antitoxin. The recipient subjects were Schick-positive (<0.001 unit antitoxin per ml. serum) and tuberculin-negative at the time of transfer. All the recipients remained Schick-positive for at least 2 weeks following transfer and in every case their serum contained less than 0.001 units antitoxin at the time when they exhibited maximal skin reactivity to toxoid. The “transfer factor” that was utilized was prepared by washing packed leukocytes isolated using the bovine fibrinogen method, and washing the leukocytes twice in recipient plasma. The washed leukocytes were subsequently lysed by 7-10 freeze-thaw cycles in the presence of DNAse with Mg++. Administration of the extract was performed intradermally and subcutaneously over the deltoid area.

Given that in those early days little was known regarding T cell specificity and MHC antigen presentation, the possibility that immunological information was transmitted by these low molecular weight transfer factors was taken seriously. Transfer factors of various sizes and charges were isolated, with some concept that different antigens elicited different types of transfer factors [277, 278]. Numerous theories were proposed to the molecular nature of transfer factor. Some evidence was that it constituted chains of antibodies that were preformed but subsequently cleaved [279]. Functionally, one of the main thoughts was that transfer factor has multiple sites of action, including effects on the thymus, on lymphocyte-monocyte and/or lymphocyte-lymphocyte interactions, as well as direct effects on cells in inflammatory sites. It is also suggested that the “specificity” of transfer factor is determined by the immunologic status of the recipient rather than by informational molecules in the dialysates [280].

Burger et al [281], used exclusion chromatography to perform characterization of transfer factor. The found that specific transferring ability of transfer factor in vivo was found to reside in the major UV-absorbing peak (Fraction III). Fraction III transferred tuberculin, candida, or KLH-reactivity to previously negative recipients. Fraction III from nonreactive donors was ineffective. When the fractions were tested in vitro, we found that both the mitogenic activity of whole transfer factor and the suppressive activity to mitogen activation when present in transfer factor was found in Fraction I. Fraction III contained components responsible for augmentation of PHA and PWM responses. In addition, Fraction III contained the component responsible for antigen-dependent augmentation of lymphocyte transformation. Fraction IV was suppressive to antigen-induced lymphocyte transformation.

In 1992 Kirkpatrick characterized the specific transfer factor at molecular level. The transfer factor is constituted by a group of numerous molecules, of low molecular weight, from 1.0 to 6.0 kDa. The 5 kDa fraction corresponds to the transfer factor specific to antigens. There are a number of publications about the clinical indications of the transfer factor for diverse diseases, in particular those where the cellular immune response is compromised or in those where there is a deficient regulation of the immune response. It has been demonstrated that the transfer factor increases the expression of IFN-gamma and RANTES, while decreases the expression of osteopontine. Using animal models it has been reported that transfer factor possesses activity against M. tuberculosis, and with a model of glioma with good therapeutic results. In the clinical setting studies have reported effects against herpes zoster, herpes simplex type I, herpetic keratitis, atopic dermatitis, osteosarcoma, tuberculosis, asthma, post-herpetic neuritis, anergic coccidioidomycosis, leishmaniasis, toxoplasmosis, mucocutaneous candidiasis, pediatric infections produced by diverse pathogen germs, sinusitis, pharyngitis, and otits media. All of these diseases were studied through protocols which main goals were to study the therapeutic effects of the transfer factor, and to establish in a systematic way diverse dosage schema and time for treatment to guide the prescription of the transfer factor [282].

In some embodiments of the invention, administration of intravenous vitamin C is utilized. Patients treated with immunotherapy have been shown to develop a scurvy-like condition. The patient presented with acute signs and symptoms of scurvy (perifollicular petechiae, erythema, gingivitis and bleeding). Serum ascorbate levels were significantly reduced to almost undetectable levels [283]. Although the role of ascorbic acid (AA) hypersupplementation in stimulation of immunity in healthy subjects is controversial, it is well established that AA deficiency is associated with impaired cell mediated immunity. This has been demonstrated in numerous studies showing deficiency suppresses T cytotoxic responses, delayed type hypersensitivity, and bacterial clearance [284]. Additionally, it is well-known that NK activity, which IL-2 is anti-tumor activity is highly dependent on, is suppressed during conditions of AA deficiency [285]. Thus it may be that while IL-2 therapy on the one hand is stimulating T and NK function, the systemic inflammatory syndrome-like effects of this treatment may actually be suppressed by induction of a negative feedback loop. Such a negative feedback loop with IL-2 therapy was successfully overcome by work using low dose histamine to inhibit IL-2 mediated immune suppression, which led to the “drug” Ceplene (histamine dichloride) receiving approval as an IL-2 adjuvant for treatment of AML [286].

The concept of AA deficiency subsequent to IL-2 therapy was reported previously by another group. Marcus et al evaluated 11 advanced cancer patients suffering from melanoma, renal cell carcinoma and colon cancer being on a 3 phase immunotherapeutic program consisting of: a) 5 days of i.v. high-dose (10(5) units/kg every 8 h) interleukin 2, (b) 6½ days of rest plus leukapheresis; and (c) 4 days of high-dose interleukin 2 plus three infusions of autologous lymphokine-activated killer cells. Mean plasma ascorbic acid levels were normal (0.64+/−0.25 mg/dl) before therapy. Mean levels dropped by 80% after the first phase of treatment with high-dose interleukin 2 alone (0.13+/−0.08 mg/dl). Subsequently plasma ascorbic acid levels remained severely depleted (0.08 to 0.13 mg/dl) throughout the remainder of the treatment, becoming undetectable (less than 0.05 mg/dl) in eight of 11 patients during this time. Importantly, blood pantothenate and plasma vitamin E remained within normal limits in all 11 patients throughout the phases of therapy, suggesting the hypovitaminosis was specific AA. Strikingly, Responders (n=3) differed from nonresponders (n=8) in that plasma ascorbate levels in the former recovered to at least 0.1 mg/dl (frank clinical scurvy) during Phases 2 and 3, whereas levels in the latter fell below this level [287]. Similar results were reported in another study by the same group examining an additional 15 patients [288]. The possibility that prognosis was related to AA levels is intriguing because of the possibility of higher immune response in these patients, however this has not been tested.

The state of AA deficiency in cancer patients, whether or not as a result of inflammation, suggests supplementation may yield benefit in quality of life. Indeed this was one of the main findings that stimulating us to write this review [289]. Improvements in quality of life were also noted in the early studies of Murata et al [290], as well as Cameron [291]. But in addition to this endpoint there appears to be a growing number of studies suggesting direct anti-cancer effects via generation of free radicals locally at tumor sites [292]. In vitro studies on a variety of cancer cells including neuroblastoma [293], bladder cancer [294], pancreatic cancer [295], mesothelioma [296], hepatoma [297], have demonstrated cytotoxic effects at pharmacologically achievable concentrations.

Enhancement of cytotoxicity of Docetaxel, Epirubicin, Irinotecan and 5-FU to a battery of tumor cell lines by AA was demonstrated in vitro [298]. In vivo studies have also supported the potential anticancer effects of AA. For example, Pollard et al used the rat PAIII androgen-independent syngeneic prostate cancer cell line to induce tumors in Lobund-Wistar rats. Daily intraperitoneal administration of AA for 30 days, with evaluation at day 40 revealed significant inhibition of tumor growth, as well as reduction in pulmonary and lymphatic metastasis [299]. Levine's group reported successful in vivo inhibition of human xenografted glioma, overian, and neuroblastoma cells in immune deficient animals by administration of AA. Interestingly control fibroblasts were not affected [300]. Clinical reports of remission induced by IV AA have been published [301], however, as mentioned above, formal trials are still ongoing.

In addition to direct cytotoxicity of AA on tumor cells, inhibition of angiogenesis may be another mechanism of action. It has been reported that AA inhibits HUVEC proliferation in vitro [302], as well as suppressing neovascularization in the chorionic allontoic membrane assay [303]. Recently we have reported that in vivo administration of AA results in suppressed vascular cord formation in mouse models [304]. Supporting this possibility, Yeom et al demonstrated that parenteral administration of AA in the S-180 sarcoma model leads to reduced tumor growth, which was associated with suppression of angiogenesis and the pro-angiogenic factors bFGF, VEGF, and MMP-2 [305]. Recent studies suggest that AA suppresses activation of the hypoxia inducible factor (HIF)-1, which is a critical transcription factor that stimulates tumor angiogenesis [306-308]. The clinical relevance of this has been demonstrated in a study showing that endometrial cancer patients having reduced tumor ascorbate levels possess higher levels of HIF-1 activation and a more aggressive phenotype [309].

Thus the possibility exists that administration of AA for treatment of tumor inflammatory mediated pathologies may also cause an antitumor effect. Whether this effect is mediated by direct tumor cytotoxicity or inhibition of angiogenesis remains to be determined. Unfortunately none of the ongoing trials of AA in cancer patients seek to address this issue [310-315].

Despite numerous claims in the popular media and even on vitamin labels, the concept of AA stimulating immunity is not as clear-cut. Part of this is because ROS are involved in numerous signals of immune cells [316]. For example, it is known that T cell receptor signaling induces an intracellular flux of ROS which is necessary for T cell activation [317]. There are numerous studies demonstrating ascorbic acid under certain conditions actually can inhibit immunity. For example, high dose ascorbate inhibits T cell and B cell proliferative responses as well as IL-2 secretion in vitro [318, 319], as well as NK cytotoxic activity [320]. Additionally, AA has been demonstrated to inhibit T cell activating ability of dendritic cells by rendering them in an immature state in part through inhibition of NF-kappa B [321].

However, It appears that the immune stimulatory effects of AA are actually observed in the context of background immune suppression or in situations of AA deficiency, both of which are well-known in the cancer and SIRS patient. A common occurrence in cancer [322-326] and SIRS patients [327, 328] is the presence of a cleaved T cell receptor (TCR) zeta chain. The zeta chain is an important component of T cell and NK cell activation, that bears the highest number of immunoreceptor tyrosine-based activation motifs (ITAMs) of other TCR and NK signaling molecules [329]. At a cellular level cleavage of the zeta chain is associated with loss of T/NK cell function and spontaneous apoptosis [330-332], at a clinical level it is associated with poor prognosis [333-338].

Since loss of TCR zeta chain is found in other inflammatory conditions ranging from hemodialysis [339, 340], to autoimmunity [341-344], to heart disease [345], the possibility that inflammatory mediators such as ROS cause TCR zeta downregulation has been suggested. Circumstantial evidence comes from studies associated inflammatory cells such as tumor associated macrophages (TAMS) with suppression of zeta chain expression [346]. Myeloid suppressor cells, which are known to produce high concentrations of ROS [347-349] have also been demonstrated to induce reduction of TCR zeta chain in cancer [350], and post trauma [351]. Administration of anti-oxidants has been shown to reverse TCR zeta chain cleavage in tissue culture [352, 353]. Therefore, from the T cell side of immunity, an argument could be made that intravenous ascorbic acid may upregulate immunity by blocking zeta chain downregulation in the context of cancer and acute inflammation.

While it is known that AA functions as an antioxidant in numerous biological conditions, as well as reduces inflammatory markers, the possibility that AA actually increases immune function in cancer patients, as well as is effects on survival and other cancer-related events, has never been formally tested. IV AA has a long and controversial history in relation to reducing tumors in patients. This has impeded research into other potential benefits of this therapy in cancer patients such as reduction of inflammation, improvement of quality of life, and impeding SIRS initiation and progression to MOF. While ongoing clinical trials of IV AA for cancer may or may not meet the bar to grant this modality a place amongst the recognized chemotherapeutic agents, it is critical that we collect as much biological data as possible, given the possibility of this agent to be a meaningful adjuvant therapy.

It is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

Various embodiments of the invention are described above in the Detailed Description. While these descriptions directly describe the above embodiments, it is understood that those skilled in the art may conceive modifications and/or variations to the specific embodiments shown and described herein. Any such modifications or variations that fall within the purview of this description are intended to be included therein as well. Unless specifically noted, it is the intention of the inventors that the words and phrases in the specification and claims be given the ordinary and accustomed meanings to those of ordinary skill in the applicable art(s).

The foregoing description of various embodiments of the invention known to the applicant at this time of filing the application has been presented and is intended for the purposes of illustration and description. The present description is not intended to be exhaustive nor limit the invention to the precise form disclosed and many modifications and variations are possible in the light of the above teachings. The embodiments described serve to explain the principles of the invention and its practical application and to enable others skilled in the art to utilize the invention in various embodiments and with various modifications as are suited to the particular use contemplated. Therefore, it is intended that the invention not be limited to the particular embodiments disclosed for carrying out the invention.

While particular embodiments of the present invention have been shown and described, it will be obvious to those skilled in the art that, based upon the teachings herein, changes and modifications may be made without departing from this invention and its broader aspects and, therefore, the appended claims are to encompass within their scope all such changes and modifications as are within the true spirit and scope of this invention.

In one embodiment the invention teaches immunization with peptides representing oncological tissue or frameshift (FS) variants of said peptides together with first altering the tumor microevironment. The invention provides a universal vaccine for administration prophylactically or therapeutically can be comprised of any of the microsatelline (MS) FS antigens. For example, one could choose all MS FS in essential genes that are greater than 30 aa long. Or one could choose all MS FS where the MS is longer than 6 nucleotides. Or one could choose a combination of criteria. One could also use the arrays of FS peptides to screen sera to determine the most often presented FS in a set of cancer types or even all cancer types. For creation of the vaccine immunotherapy binding to MHC is required for T cell activity and can be determined by binding assays. Alternatively, in silico methods of MHC binding are used to predict binding of a peptide to a MHC subtype. Data of peptides binding to MHC subtype molecules are used to develop binding prediction algorithms. These algorithms calculate scoring matrices that quantify the contribution of each residue in a fixed-length peptide to binding to an MHC molecule. Algorithms predict binding of a peptide to class I MHC or class II MHC. Algorithms to predict class I MHC binding include but are not limited to Artificial neural network (ANN), Stabilized matrix method (SMM), SMM with a Peptide:MHC Binding Energy Covariance matrix (SMMPMBEC), Scoring Matrices derived from Combinatorial Peptide Libraries (Comblib_Sidney2008), Consensus, NetMHCpan, NetMHCcons and PickPocket. Algorithms to predict class II MHC binding, include but are not limited to Consensus method, Combinatorial library, NN-align (netMHCII-2.2), SMM-align (netMHCII-1.1), Sturniolo, and NetMHCIIpan. The entire population of FS polypeptides is then scanned using one or more of the above algorithms for peptides binding to an MHC subtype molecule with a predicted affinity of IC50<500 nM.

In some embodiments, candidate frameshift (FS) peptides are screened for T cell activity. T cell activity is determined using a T cell assay measuring proliferation, cytokine secretion, cytotoxicity, or degranulation in response to a FS peptide bound to an antigen presenting cell. T cell assays include but are not limited to proliferation assay, 3H-thymidine assay, BrdU assay, CFSE assay, cytokine secretion assay, ELISA assay, ELISPOT assay, intracellular staining assay, quantitative rtPCR assay, cytometric bead array assay, MHC-tetramer binding assay, cytotoxicity assay, 51-chromium assay, degranulation assay, granulysin assay, granzyme A assay, granzyme B assay, and perforin assay. In an exemplary embodiment, a blood sample is obtained from an individual. PBMCs are isolated from the blood sample and the PBMCs are cultured to expand T cells in the sample and the T cells are incubated in culture media containing one or more candidate peptides for a cytokine release assay. The production of IFN-.gamma. is analyzed in ELISPOT assays. Flat-bottom 96-well nitrocellulose plates are prepared and coated with either anti-human IFN-.gamma.. Cells were then incubated at a density of 1.times.10.sup.5/well either with peptide pools or individual peptides (10.mu.g/ml), PHA (10 .mu.g/ml), or medium (containing 1% DMSO corresponding to the percentage of DMSO in the pools/peptides) as a control. After 24 hours, cells are removed, and plates are incubated with HRP-conjugated anti-human IFN-.gamma. Ab (Clone 7-B6-1, Mabtech) at 37.degree. C. After 2 hours, spots corresponding to the HRP-conjugated Ab (IFN-.gamma.) are developed with 3-amino-9-ethylcarvazole solution (Sigma-Aldrich, St. Louis, Mo.). Spots are counted by computer-assisted image analysis (Zeiss, KS-ELISPOT reader, Munich, Germany). Each assay is performed in triplicate. The level of statistical significance is determined with a Student's t-test using the mean of triplicate values of the response against relevant pools or individual peptides versus the response against the DMSO control. Criteria for peptide pool positivity are 100 spot-forming cells (SFCs)/10.sup.6 PBMC, p.ltoreq.0.05 and a stimulation index (SI).gtoreq.2, while criteria for individual peptide positivity are .gtoreq.20 SFC/10.sup.6 PBMC, p.ltoreq.0.05, and a SI. gtoreq.2. The peptide arrays can include control sequences that match epitopes of well characterized monoclonal antibodies (mAbs). Binding patterns to control sequences and to library peptides can be measured to qualify the arrays and the assay process. Additionally, inter wafer signal precision can be determined by testing sample replicates e.g. plasma samples, on arrays from different wafers and calculating the coefficients of variation (CV) for all library peptides. Precision of the measurements of binding signals can be determined as an aggregate of the inter-array, inter-slide, inter-wafer and inter-day variations made on arrays synthesized on wafers of the same batch (within wafer batches). Additionally, precision of measurements can be determined for arrays on wafers of different batches (between wafer batches). In some embodiments, measurements of binding signals can be made within and/or between wafer batches with a precision varying less than 5%, less than 10%, less than 15%, less than 20%, less than 25%, or less than 30%. The technologies disclosed herein include a photolithographic array synthesis platform that merges semiconductor manufacturing processes and combinatorial chemical synthesis to produce array-based libraries on silicon wafers. By utilizing the tremendous advancements in photolithographic feature patterning, the array synthesis platform is highly-scalable and capable of producing combinatorial peptide libraries with 40 million features on an 8-inch wafer. Photolithographic array synthesis is performed using semiconductor wafer production equipment in a class 10,000 cleanroom to achieve high reproducibility. When the wafer is diced into standard microscope

Numerous embodiments are further provided that can be applied to any aspect of the present invention and/or combined with any other embodiment described herein. For example, in one embodiment, the cancer cells have reduced copy number, amount, and/or activity of one or more DNA damage checkpoints and/or DNA damage repair genes. In another embodiment, the one or more DNA damage checkpoints are selected from the group consisting of Brca1, Brca2, Chk1, Chk2, ATM, ATR, Cdc25C, and Nbs1. In still another embodiment, the one or more DNA damage repair genes are selected from the group consisting of non-homologous end joining (NHEJ), microhomology-mediated end joining (MMEJ), and homologous recombination pathway genes. For example, the one or more DNA damage repair genes can be selected from the group consisting of BLM, MSH2, MSH6, MLH1, PMS2, MRE11, DNA Ligase IV, TP53BP1, RAD51, RAD51L1, RAD51C, RAD51L3, DMC1, XRCC2, XRCC3, XRCC4, NBS1, RAD50, GADD45, RFC2, XRCC6, POLD2, PCNA, RPA1, RPA2, ERCC3, UNG, ERCC5, MLH1, LIG1, NBN, MSH6, POLD4, RFC5, DDB2, POLD1, FANCG, POLB, XRCC1, MPG, RFC2, ERCC1, TDG, FANCA, RFC4, RFC3, APEX2, RAD1, BRCA1, FEN1, MLH3, MGMT, RAD51, XRCC4, RECQL, ERCC8, FANCC, OGG1, MRE11A, RAD52, WRN, XPA, BLM, OGG1, MSH3, POLE2, RAD51C, LIG4, ERCC6, LIG3, RAD17, XRCC2, MUTYH, RFC1, BRCA2, RAD50, DDB1, XRCC5, PARP1, POLE3, RFC1, RAD50, XPC, MSH2, RPA3, MBD4, NTHL1, PMS2/PMS2CL, RAD51C, UNG2, APEX1, ERCC4, RAD1, RECQL5, MSH5, RECQL, RAD52, XRCC4, RAD17, MSH3, MRE11A, MSH6, and RECQL5. In yet another embodiment, the copy number, amount, and/or activity of one or more DNA damage checkpoints and/or DNA damage repair genes are reduced by contacting the cancer cells with a small molecule inhibitor, CRISPR guide RNA (gRNA), RNA interfering agent, antisense oligonucleotide, peptide or peptidomimetic inhibitor, aptamer, antibody, or intrabody. In one embodiment, the RNA interfering agent can be a small interfering RNA (siRNA), CRISPR RNA (crRNA), a CRISPR guide RNA (gRNA), a small hairpin RNA (shRNA), a microRNA (miRNA), or a piwi-interacting RNA (piRNA). In another embodiment, the antibody and/or intrabody, or antigen binding fragment thereof, specifically binds to one or more DNA damage checkpoints and/or DNA damage repair genes. In still another embodiment, the antibody and/or intrabody, or antigen binding fragment thereof, is murine, chimeric, humanized, composite, or human. In yet another embodiment, the antibody and/or intrabody, or antigen binding fragment thereof, comprises an effector domain, comprises an Fc domain, and/or is selected from the group consisting of Fv, Fav, F(ab′)2, Fab′, dsFv, scFv, sc(Fv)2, and diabodies fragments.

In some embodiments various adjuvants or excipients are utilized together riwht immunotherapyu. Vaccine compositions herein, in some embodiments, comprise a pharmaceutically acceptable adjuvant or excipient. In some embodiments, the adjuvant is selected from the group consisting of ABM2, AS01B, AS02, AS02A, Adjumer, Adjuvax, Algammulin, Alum, Aluminum phosphate, Aluminum potassium sulfate, Bordetella pertussis, Calcitriol, Chitosan, Cholera toxin, CpG, Dibutyl phthalate, Dimethyldioctadecylammonium bromide (DDA), Freund's adjuvant, Freund's complete, Freund's incomplete (IFA), GM-CSF, GMDP, Gamma Inulin, Glycerol, HBSS (Hank's Balanced Salt Solution), IL-12, IL-2, Imiquimod, Interferon-Gamma, ISCOM, Lipid Core Peptide (LCP), Lipofectin, Lipopolysaccharide (LPS), Liposomes, MF59, MLP+TDM, Monophosphoryl lipid A, Montanide IMS-1313, Montanide ISA 206, Montanide ISA 720, Montanide ISA-51, Montanide ISA-50, nor-MDP, Oil-in-water emulsion, P1005 (non-ionic copolymer), Pam3Cys (lipoprotein), Pertussis toxin, Poloxamer, QS21, RaLPS, Ribi, Saponin, Seppic ISA 720, Soybean Oil, Squalene, Syntex Adjuvant Formulation (SAF), Synthetic polynucleotides (poly IC/poly AU), TiterMax Tomatine, Vaxfectin, XtendIII, and Zymosan. Vaccine compositions herein, in some embodiments, are administered via a route selected from the group consisting of subcutaneous, intradermal, intramuscular, intranasal, intravenous, and sublingual. Individuals in need of administration of a universal vaccine, in some embodiments are mammals. In some embodiments, the individual is a human, a dog, a cat, a mouse, a rat, a rabbit, a horse, a cow, or a pig.

The methods and treatment protocols described herein comprise administration of immunotherapies and preparative protocols in an individual. In some embodiments, the individual is diagnosed with cancer. In some embodiments, the individual has increased risk of developing cancer. In some embodiments, the cancer comprises Acanthoma, Acinic cell carcinoma, Acoustic neuroma, Acral lentiginous melanoma, Acrospiroma, Acute eosinophilic leukemia, Acute lymphoblastic leukemia, Acute megakaryoblastic leukemia, Acute monocytic leukemia, Acute myeloblastic leukemia with maturation, Acute myeloid dendritic cell leukemia, Acute myeloid leukemia, Acute promyelocytic leukemia, Adamantinoma, Adenocarcinoma, Adenoid cystic carcinoma, Adenoma, Adenomatoid odontogenic tumor, Adrenocortical carcinoma, Adult T-cell leukemia, Aggressive NK-cell leukemia, AIDS-Related Cancers, AIDS-related lymphoma, Alveolar soft part sarcoma, Ameloblastic fibroma, Anal cancer, Anaplastic large cell lymphoma, Anaplastic thyroid cancer, Angioimmunoblastic T-cell lymphoma, Angiomyolipoma, Angiosarcoma, Appendix cancer, Astrocytoma, Atypical teratoid rhabdoid tumor, Basal cell carcinoma, Basal-like carcinoma, B-cell leukemia, B-cell lymphoma, Bellini duct carcinoma, Biliary tract cancer, Bladder cancer, Blastoma, Bone Cancer, Bone tumor, Brain Stem Glioma, Brain Tumor, Breast Cancer, Brenner tumor, Bronchial Tumor, Bronchioloalveolar carcinoma, Brown tumor, Burkitt's lymphoma, Cancer of Unknown Primary Site, Carcinoid Tumor, Carcinoma, Carcinoma in situ, Carcinoma of the penis, Carcinoma of Unknown Primary Site, Carcinosarcoma, Castleman's Disease, Central Nervous System Embryonal Tumor, Cerebellar Astrocytoma, Cerebral Astrocytoma, Cervical Cancer, Cholangiocarcinoma, Chondroma, Chondrosarcoma, Chordoma, Choriocarcinoma, Choroid plexus papilloma, Chronic Lymphocytic Leukemia, Chronic monocytic leukemia, Chronic myelogenous leukemia, Chronic Myeloproliferative Disorder, Chronic neutrophilic leukemia, Clear-cell tumor, Colon Cancer, Colorectal cancer, Craniopharyngioma, Cutaneous T-cell lymphoma, Degos disease, Dermatofibrosarcoma protuberans, Dermoid cyst, Desmoplastic small round cell tumor, Diffuse large B cell lymphoma, Dysembryoplastic neuroepithelial tumor, Embryonal carcinoma, Endodermal sinus tumor, Endometrial cancer, Endometrial Uterine Cancer, Endometrioid tumor, Enteropathy-associated T-cell lymphoma, Ependymoblastoma, Ependymoma, Epithelioid sarcoma, Erythroleukemia, Esophageal cancer, Esthesioneuroblastoma, Ewing Family of Tumor, Ewing Family Sarcoma, Ewing's sarcoma, Extracranial Germ Cell Tumor, Extragonadal Germ Cell Tumor, Extrahepatic Bile Duct Cancer, Extramammary Paget's disease, Fallopian tube cancer, Fetus in fetu, Fibroma, Fibrosarcoma, Follicular lymphoma, Follicular thyroid cancer, Gallbladder Cancer, Gallbladder cancer, Ganglioglioma, Ganglioneuroma, Gastric Cancer, Gastric lymphoma, Gastrointestinal cancer, Gastrointestinal Carcinoid Tumor, Gastrointestinal Stromal Tumor, Gastrointestinal stromal tumor, Germ cell tumor, Germinoma, Gestational choriocarcinoma, Gestational Trophoblastic Tumor, Giant cell tumor of bone, Glioblastoma multiforme, Glioma, Gliomatosis cerebri, Glomus tumor, Glucagonoma, Gonadoblastoma, Granulosa cell tumor, Hairy Cell Leukemia, Hairy cell leukemia, Head and Neck Cancer, Head and neck cancer, Heart cancer, Hemangioblastoma, Hemangiopericytoma, Hemangiosarcoma, Hematological malignancy, Hepatocellular carcinoma, Hepatosplenic T-cell lymphoma, Hereditary breast-ovarian cancer syndrome, Hodgkin Lymphoma, Hodgkin's lymphoma, Hypopharyngeal Cancer, Hypothalamic Glioma, Inflammatory breast cancer, Intraocular Melanoma, Islet cell carcinoma, Islet Cell Tumor, Juvenile myelomonocytic leukemia, Kaposi Sarcoma, Kaposi's sarcoma, Kidney Cancer, Klatskin tumor, Krukenberg tumor, Laryngeal Cancer, Laryngeal cancer, Lentigo maligna melanoma, Leukemia, Lip and Oral Cavity Cancer, Liposarcoma, Lung cancer, Luteoma, Lymphangioma, Lymphangiosarcoma, Lymphoepithelioma, Lymphoid leukemia, Lymphoma, Macroglobulinemia, Malignant Fibrous Histiocytoma, Malignant fibrous histiocytoma, Malignant Fibrous Histiocytoma of Bone, Malignant Glioma, Malignant Mesothelioma, Malignant peripheral nerve sheath tumor, Malignant rhabdoid tumor, Malignant triton tumor, MALT lymphoma, Mantle cell lymphoma, Mast cell leukemia, Mediastinal germ cell tumor, Mediastinal tumor, Medullary thyroid cancer, Medulloblastoma, Medulloepithelioma, Melanoma, Meningioma, Merkel Cell Carcinoma, Mesothelioma, Metastatic Squamous Neck Cancer with Occult Primary, Metastatic urothelial carcinoma, Mixed Mullerian tumor, Monocytic leukemia, Mouth Cancer, Mucinous tumor, Multiple Endocrine Neoplasia Syndrome, Multiple myeloma, Mycosis Fungoides, Myelodysplastic Disease, Myelodysplastic Syndromes, Myeloid leukemia, Myeloid sarcoma, Myeloproliferative Disease, Myxoma, Nasal Cavity Cancer, Nasopharyngeal Cancer, Nasopharyngeal carcinoma, Neoplasm, Neurinoma, Neuroblastoma, Neurofibroma, Neuroma, Nodular melanoma, Non-Hodgkin lymphoma, Nonmelanoma Skin Cancer, Non-Small Cell Lung Cancer, Ocular oncology, Oligoastrocytoma, Oligodendroglioma, Oncocytoma, Optic nerve sheath meningioma, Oral cancer, Oropharyngeal Cancer, Osteosarcoma, Ovarian cancer, Ovarian Epithelial Cancer, Ovarian Germ Cell Tumor, Ovarian Low Malignant Potential Tumor, Paget's disease of the breast, Pancoast tumor, Pancreatic cancer, Papillary thyroid cancer, Papillomatosis, Paraganglioma, Paranasal Sinus Cancer, Parathyroid Cancer, Penile Cancer, Perivascular epithelioid cell tumor, Pharyngeal Cancer, Pheochromocytoma, Pineal Parenchymal Tumor of Intermediate Differentiation, Pineoblastoma, Pituicytoma, Pituitary adenoma, Pituitary tumor, Plasma Cell Neoplasm, Pleuropulmonary blastoma, Polyembryoma, Precursor T-lymphoblastic lymphoma, Primary central nervous system lymphoma, Primary effusion lymphoma, Primary Hepatocellular Cancer, Primary Liver Cancer, Primary peritoneal cancer, Primitive neuroectodermal tumor, Prostate cancer, Pseudomyxoma peritonei, Rectal Cancer, Renal cell carcinoma, Respiratory Tract Carcinoma Involving the NUT Gene on Chromosome 15, Retinoblastoma, Rhabdomyoma, Rhabdomyosarcoma, Richter's transformation, Sacrococcygeal teratoma, Salivary Gland Cancer, Sarcoma, Schwannomatosis, Sebaceous gland carcinoma, Secondary neoplasm, Seminoma, Serous tumor, Sertoli-Leydig cell tumor, Sex cord-stromal tumor, Sezary Syndrome, Signet ring cell carcinoma, Skin Cancer, Small blue round cell tumor, Small cell carcinoma, Small Cell Lung Cancer, Small cell lymphoma, Small intestine cancer, Soft tissue sarcoma, Somatostatinoma, Soot wart, Spinal Cord Tumor, Spinal tumor, Splenic marginal zone lymphoma, Squamous cell carcinoma, Stomach cancer, Superficial spreading melanoma, Supratentorial Primitive Neuroectodermal Tumor, Surface epithelial-stromal tumor, Synovial sarcoma, T-cell acute lymphoblastic leukemia, T-cell large granular lymphocyte leukemia, T-cell leukemia, T-cell lymphoma, T-cell prolymphocytic leukemia, Teratoma, Terminal lymphatic cancer, Testicular cancer, Thecoma, Throat Cancer, Thymic Carcinoma, Thymoma, Thyroid cancer, Transitional Cell Cancer of Renal Pelvis and Ureter, Transitional cell carcinoma, Urachal cancer, Urethral cancer, Urogenital neoplasm, Uterine sarcoma, Uveal melanoma, Vaginal Cancer, Verner Morrison syndrome, Verrucous carcinoma, Visual Pathway Glioma, Vulvar Cancer, Waldenstrom's macroglobulinemia, Warthin's tumor, or Wilms' tumor.

The concept of treating cancer by blocking new blood vessel formation, angiogenesis, was pioneered by Judah Folkman who provided convincing arguments that it is not necessary to actively kill the tumor mass, but by suppressing its ability to grow through cutting off blood supply, malignant tumors may be converted into benign masses that eventually regress [354, 355]. Unfortunately, despite discovery of angiostatin, and endostatin, naturally derived inhibitors of angiogenesis, neither of these approaches translated into successful therapies¹. Nevertheless, the concept of targeting new blood vessel formation led to thousands of publications describing various antiangiogenic agents, of which several eventually proceeded through clinical trials and regulatory approval. Broadly anti-angiogenic agents approved by regulators can be classified into antibodies, such as Bevacizumab (Avastin) which binds VEGF [356], and Ramucirumab (Cyramza) [357], which binds VEGF-R2, as well as small molecules which bind multiple receptor kinases associated with angiogenesis such as Sunitinib [358-360], Cabozantinib [361-364], Pazopanib [365-367], and Regorafenib [368-370]. ¹ http://www.nytimes.com/1998/11/13/us/a-failure-to-verify-a-cancer-advance-is-raising-concern.html

These approaches have augmented the standard of care for various tumor types and have achieved some level of progress. Unfortunately, the concept of blocking angiogenesis of cancer was not as simple as originally envisioned. One of the major hurdles in blocking angiogenesis was that even though de novo blood vessels are derived from nonmalignant cells, the malignant cells appear to possess ability to induce mutations in the new blood vessels. One example of the heterogeneity of tumor endothelial cells compared to endothelial cells from low and high metastatic tumors by Ohga et al [371]. The investigators extracted two types of tumor endothelial cells (TEM) from high-metastatic (HM) and low-metastatic (LM) tumors and compared their characteristics. HM tumor-derived TECs (HM-TECs) showed higher proliferative activity and invasive activity than LM tumor-derived TECs (LM-TECs). Moreover, the mRNA expression levels of pro-angiogenic genes, such as vascular endothelial growth factor (VEGF) receptors 1 and 2, VEGF, and hypoxia-inducible factor-1α, were higher in HM-TECs than in LM-TECs. The tumor blood vessels themselves and the surrounding area in HM tumors were exposed to hypoxia. Furthermore, HM-TECs showed higher mRNA expression levels of the stemness-related gene stem cell antigen and the mesenchymal marker CD90 compared with LM-TECs. HM-TECs were spheroid, with a smoother surface and higher circularity in the stem cell spheroid assay. HM-TECs differentiated into osteogenic cells, expressing activated alkaline phosphatase in an osteogenic medium at a higher rate than either LM-TECs or normal ECs. Furthermore, HM-TECs contained more aneuploid cells than LM-TECs. The investigators concluded that the results indicate that TECs from HM tumors have a more pro-angiogenic phenotype than those from LM tumors. It appears that the aggressiveness of the tumor not only can alter endothelial cell function but also drug resistance ability. In another study, Akiyama et al. [372]compared murine TECs and normal ECs. It was found that TECs were more resistant to paclitaxel with the up-regulation of multidrug resistance (MDR) 1 mRNA, which encodes the P-glycoprotein, compared with normal ECs. Normal human microvascular ECs were cultured in tumor-conditioned medium (CM) and became more resistant to paclitaxel through MDR1 mRNA up-regulation and nuclear translocation of Y-box-binding protein 1, which is an MDR1 transcription factor. Vascular endothelial growth factor (VEGF) receptor 2 (VEGFR2) and Akt were activated in human microvascular ECs by tumor CM. The investigators observed that tumor CM contained a significantly high level of VEGF. A VEGFR kinase inhibitor, Ki8751, and a phosphatidylinositol 3-kinase-Akt inhibitor, LY294002, blocked tumor CM-induced MDR1 up-regulation. MDR1 up-regulation, via the VEGF-VEGFR pathway in the tumor microenvironment, is one of the mechanisms of drug resistance acquired by TECs. It was observed that VEGF secreted from tumors up-regulated MDR1 through the activation of VEGFR2 and Akt. This process is a novel mechanism of the acquisition of drug resistance by TECs in the tumor microenvironment. Yet another study demonstrated that tumors can induce a “dedifferentiation” of tumor endothelium. Specifically, compared with NECs, stem cell markers such as Sca-1, CD90, and multidrug resistance 1 are upregulated in TECs, suggesting that stem-like cells exist in tumor blood vessels. TECs and NECs were isolated from melanoma-xenografted nude mice and normal dermis, respectively. The stem cell marker aldehyde dehydrogenase (ALDH) mRNA expression and activity were higher in TECs than those in NECs. Next, ALDHhigh/low TECs were isolated by fluorescence-activated cell sorting to compare their characteristics. Compared with ALDHlow TECs, ALDHhigh TECs formed more tubes on Matrigel-coated plates and sustained the tubular networks longer. Furthermore, VEGFR2 expression was higher in ALDHhigh TECs than that in ALDHlow TECs. In addition, ALDH was expressed in the tumor blood vessels of in vivo mouse models of melanoma and oral carcinoma, but not in normal blood vessels. These findings indicate that ALDHhigh TECs exhibit an angiogenic phenotype. Stem-like TECs may have an essential role in tumor angiogenesis [373].

What is it that causes the tumor to evoke changes in the endothelium? As suggested above, there is some support for growth factor mediated alterations, additionally, horizontal gene transfer may also play a role [374-382]. Although the field of horizontal gene transfer has historically been controversial one of the strongest evidences supporting this concept is the phenomena of donor-derived relapse in leukemic patients. In these situations patients with leukemia who relapse after bone marrow transplant have the relapsing cells originate from donor cells that transformed into malignant cells [383, 384]. Another issue that affected efficacy of anti-angiogenesis therapies is that in some tumors, the tumor cells themselves transdifferentiate into endothelial-like cells, termed tumor vascular channels, which possess ability to mutate around either antibody or kinase inhibitor drugs [385-390].

The previously mentioned means by which tumor endothelial cells can protect themselves against anti-angiogenic agents has resulted in relatively low clinical efficacy of these drugs. To understand the general lack of efficacy in the initial registration trial², median progression free survival (PFS) of ovarian cancer patients who received bevacizumab plus chemotherapy was 6.8 months (95 percent CI: 5.6, 7.8) compared with 3.4 months (95 percent CI: 2.1, 3.8) for those who received chemotherapy alone. There was no statistically significant difference in overall survival (OS) for patients treated with bevacizumab plus chemotherapy compared with chemotherapy alone (median OS: 16.6 months versus 13.3 months; HR 0.89; 95 percent CI: 0.69, 1.14). Subset analysis led to identification that the group of patients that received paclitaxel with the antibody had the largest improvement, resulting in a 5.7-month improvement in median PFS (9.6 months versus 3.9 months; HR 0.47; 95 percent CI: 0.31, 0.72), an improvement in the objective response rate (53 percent versus 30 percent), and a 9.2-month improvement in median OS (22.4 months versus 13.2 months, HR 0.64; 95 percent CI: 0.41, 1.01)³. Multiple other trials where conducted for different indications using bevacizumab, unfortunately, progression free survival and overall survival was not increase more than a year in any of the studies [391-395], and neither in studies with small molecule kinase inhibitors [396-401]. ² https://clinicaltrials.gov/show/NCT00976911³ https://www.cancer.gov/about-cancer/treatment/drugs/fda-bevacizumab

This clinical translation, although highly beneficial in some patients, overall the effect was mediocre, highlights the disparity between animal studies, in which some studies complete regression was observed in established tumors [402, 403], whereas in clinical trials, relatively minimal effect compared to animal studies was observed [404]. One lesson from these studies is that the large heterogeneity of the patient and of the tumors, which calls for large patient populations in order to achieve an overall survival advantage. Innovations in pharmacogenomics and personalized medicine will help identify specific patients and tumors that are likely to respond. Unfortunately, at present, patients with metastatic disease have limited options and a statistically significant extension of survival does equate to large market demand, as seen by the overall sale of angiogenesis inhibitors for cancer being over 20 billion annually.

One embodiment of the invention is a short-interfering ribonucleic acid (siRNA) molecule effective at silencing NR2F6 expression or substantially inhibiting NR2F6 expression. In one embodiment of the invention the oligonucleotide backbone is chemically modified to increase the deliverability of the interfering ribonucleic acid molecule. In another embodiment these chemical modifications act to neutralize the negative charge of the interfering ribonucleic acid molecule. One embodiment of the invention consists of a pharmaceutical composition comprising an siRNA oligonucleotide that induces RNA interference against NR2F6. It is known to one of skill in the art that siRNAs induce a sequence-specific reduction in expression of a gene by the process of RNAi, as previously mentioned. Thus, siRNA is the intermediate effector molecule of the RNAi process that is normally induced by double stranded viral infections, with the longer double stranded RNA being cleaved by naturally occurring enzymes such as DICER. Some nucleic acid molecules or constructs provided herein include double stranded RNA molecules comprising 16-30, e.g., 16, 17, 18, 19, 20, 21, 22, 23, 24, 25, 26, 27, 28, 29, or 30 nucleotides in each strand, wherein one of the strands is substantially identical, for example at least 85% (or more, as for example, 90%, 95%, or 100%) identical, e.g., having 3, 2, 1, or 0 mismatched nucleotide(s), to a target region in the mRNA of NR2F6 and the other strand is identical or substantially identical to the first strand. However, it will be appreciated that the dsRNA molecules may have any number of nucleotides in each strand which allows them to reduce the level of NR2F6 protein, or the level of a nucleic acid encoding NR2F6. The dsRNA molecules provided herein can be chemically synthesized, or can be transcribed in vitro from a DNA template, or in vivo from, e.g., shRNA, which is mentioned below. The dsRNA molecules can be designed using any method known in the art.

In one embodiment, nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single crosslink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F6. In some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F6 gene, such as a nucleic acid encoding the NR2F6 mRNA, and can be driven, for example, by separate Pol III promoter sites. In some situations it will be preferable to induce expression of the hairpin siRNA or shRNAs in a tissue specific manner in order to activate the shRNA transcription that would subsequently silence NR2F6 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues. For example, promoters such as the PSA, probasin, prostatic acid phosphatase or prostate-specific glandular kallikrein (hK2) may be used to target gene expression in the prostate. Similarly, promoters as follows may be used to target gene expression in other tissues. Examples of more tissue specific promoters include in (a) to target the pancreas promoters for the following may be used: insulin, elastin, amylase, pdr-I, pdx-I, glucokinase; (b) to target the liver promoters for the following may be used: albumin PEPCK, HBV enhancer, a fetoprotein, apolipoprotein C, .alpha.-I antitrypsin, vitellogenin, NF-AB, Transthyretin; (c) to target the skeletal muscle promoters for the following may be used: myosin H chain, muscle creatine kinase, dystrophin, calpain p94, skeletal .alpha.-actin, fast troponin 1; (d) to target the skin promoters for the following may be used: keratin K6, keratin KI; (e) lung: CFTR, human cytokeratin IS (K 18), pulmonary surfactant proteins A, B and C, CC-10, Pi; (0 smooth muscle: sm22.alpha., SM-.alpha.-actin; (g) to target the endothelium promoters for the following may be used: endothelin-I, E-selectin, von Willebrand factor, TIE, KDR/flk-I; (h) to target melanocytes the tyrosinase promoter may be used; (i) to target the mammary gland promoters for the following may be used: MMTV, and whey acidic protein (WAP).

Yet another embodiment of the invention consists of a pharmaceutical composition comprising an oligonucleotide that induces RNA interference against NR2F6 combined with a delivery agent such as a liposome. For more targeted delivery immunoliposomes, or liposomes containing an agent inducing selective binding to neoplastic cells may be used.

The present invention further provides pharmaceutical compositions comprising the nucleic acid-lipid particles described herein and a pharmaceutically acceptable carrier.

Another embodiment of the invention consists of a pharmaceutical composition comprising an oligonucleotide that induces RNA interference against NR2F6 combined with an additional chemotherapeutic agent.

Yet another embodiment of the invention consists of a pharmaceutical composition comprising an oligonucleotide that induces RNA interference against NR2F6 combined with an additional agent used to induce differentiation of endothelial cells associated with cancer.

One embodiment of the invention is a short-interfering ribonucleic acid (siRNA) molecule effective at silencing NR2F6 expression that has been cloned into an appropriate expression vector giving rise to an shRNA vector.

In certain embodiment shRNA olignucleotides are cloned into an appropriate mammalian expression vectors, examples of appropriate vectors include but are not limited to lentiviral, retroviral or adenoviral vector.

As used herein, the term “target nucleic acid” encompasses DNA, RNA (comprising premRNA and mRNA) transcribed from such DNA, and also cDNA derived from such RNA, coding, noncoding sequences, sense or antisense polynucleotides. The specific hybridization of an oligomeric compound with its target nucleic acid interferes with the normal function of the nucleic acid. This modulation of function of a target nucleic acid by compounds, which specifically hybridize to it, is generally referred to as “antisense”. The functions of DNA to be interfered include, for example, replication and transcription. The functions of RNA to be interfered, include all vital functions such as, for example, translocation of the RNA to the site of protein translation, translation of protein from the RNA, splicing of the RNA to yield one or more mRNA species, and catalytic activity which may be engaged in or facilitated by the RNA. The overall effect of such interference with target nucleic acid function is modulation of the expression of an encoded product or oligonucleotides.

For the purpose of the invention, suppression of NR2F6 is performed in endothelial cells associated with pathology such as in cancer or wet macular degeneration. RNA interference “RNAi” is mediated by double stranded RNA (dsRNA) molecules that have sequence-specific homology to their “target” nucleic acid sequences (Caplen, N. J., et al. (2001) Proc. Natl. Acad. Sci. USA 98:9742-9747). In certain embodiments of the present invention, the mediators are 5-25 nucleotide “small interfering” RNA duplexes (siRNAs). The siRNAs are derived from the processing of dsRNA by an RNase enzyme known as Dicer (Bernstein, E., et al. (2001) Nature 409:363-366). siRNA duplex products are recruited into a multi-protein siRNA complex termed RISC (RNA Induced Silencing Complex). Without wishing to be bound by any particular theory, a RISC is then believed to be guided to a target nucleic acid (suitably mRNA), where the siRNA duplex interacts in a sequence-specific way to mediate cleavage in a catalytic fashion (Bernstein, E., et al. (2001) Nature 409:363-366; Boutla, A., et al. (2001) Curr. Biol. 11:1776-1780). Small interfering RNAs that can be used in accordance with the present invention can be synthesized and used according to procedures that are well known in the art and that will be familiar to the ordinarily skilled artisan. Small interfering RNAs for use in the methods of the present invention suitably comprise between about 1 to about 50 nucleotides (nt). In examples of non limiting embodiments, siRNAs can comprise about 5 to about 40 nt, about 5 to about 30 nt, about 10 to about 30 nt, about 15 to about 25 nt, or about 20-25 nucleotides.

Selection of appropriate oligonucleotides is facilitated by using computer programs that automatically align nucleic acid sequences and indicate regions of identity or homology. Such programs are used to compare nucleic acid sequences obtained, for example, by searching databases such as GenBank or by sequencing PCR products. Comparison of nucleic acid sequences from a range of species allows the selection of nucleic acid sequences that display an appropriate degree of identity between species. In the case of genes that have not been sequenced, Southern blots are performed to allow a determination of the degree of identity between genes in target species and other species. By performing Southern blots at varying degrees of stringency, as is well known in the art, it is possible to obtain an approximate measure of identity. These procedures allow the selection of oligonucleotides that exhibit a high degree of complementarity to target nucleic acid sequences in a subject to be controlled and a lower degree of complementarity to corresponding nucleic acid sequences in other species. One skilled in the art will realize that there is considerable latitude in selecting appropriate regions of genes for use in the present invention.

The term “nucleotide” covers naturally occurring nucleotides as well as nonnaturally occurring nucleotides. It should be clear to the person skilled in the art that various nucleotides which previously have been considered “non-naturally occurring” have subsequently been found in nature. Thus, “nucleotides” includes not only the known purine and pyrimidine heterocycles-containing molecules, but also heterocyclic analogues and tautomers thereof. Illustrative examples of other types of nucleotides are molecules containing adenine, guanine, thymine, cytosine, uracil, purine, xanthine, diaminopurine, 8-oxo-N6-methyladenine, 7-deazaxanthine, 7-deazaguanine, N4,N4-ethanocytosin, N6,N6-ethano-2,6-diaminopurine, 5-methylcytosine, 5-(C3-C6)-alkynylcytosine, 5-fluorouracil, 5-bromouracil, pseudoisocytosine, 2-hydroxy-5-methyl-4-triazolopyridin, isocytosine, isoguanin, inosine and the “non-naturally occurring” nucleotides described in Benner et al., U.S. Pat. No. 5,432,272. The term “nucleotide” is intended to cover every and all of these examples as well as analogues and tautomers thereof. Especially interesting nucleotides are those containing adenine, guanine, thymine, cytosine, and uracil, which are considered as the naturally occurring nucleotides in relation to therapeutic and diagnostic application in humans. Nucleotides include the natural 2′-deoxy and 2′-hydroxyl sugars, e.g., as described in Komberg and Baker, DNA Replication, 2nd Ed. (Freeman, San Francisco, 1992) as well as their analogs.

As used herein, the term “cancer” refers to any malignant tumor, particularly arising in the lung, kidney, or thyroid. The cancer manifests itself as a “tumor” or tissue comprising malignant cells of the cancer. Examples of tumors include sarcomas and carcinomas such as, but not limited to: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma. As noted above, the invention specifically permits differential diagnosis of lung, kidney, and thyroid tumors.

According to the present invention, antisense compounds include antisense oligonucleotides, ribozymes, external guide sequence (EGS) oligonucleotides, siRNA compounds, single- or double-stranded RNA interference (RNAi) compounds such as siRNA compounds, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, doublestranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleotide positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines, however, in some embodiments, the gene expression or function is up regulated. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

Once introduced to a system, the compounds of the invention may elicit the action of one or more enzymes or structural proteins to effect cleavage or other modification of the target nucleic acid or may work via occupancy-based mechanisms. In general, nucleic acids (including oligonucleotides) may be described as “DNA-like” (i.e., generally having one or more 2′-deoxy sugars and, generally, T rather than U bases) or “RNA-like” (i.e., generally having one or more 2′-hydroxyl or 2′-modified sugars and, generally U rather than T bases). Nucleic acid helices can adopt more than one type of structure, most commonly the A- and B-forms. It is believed that, in general, oligonucleotides which have B-form-like structure are “DNA-like” and those which have A-formlike structure are “RNA-like.” In some (chimeric) embodiments, an antisense compound may contain both A- and B-form regions.

In another preferred embodiment, the desired oligonucleotides or antisense compounds, comprise at least one of: antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof. dsRNA can also activate gene expression, a mechanism that has been termed “small RNA-induced gene activation” or RNAa. dsRNAs targeting gene promoters induce potent transcriptional activation of associated genes. RNAa was demonstrated in human cells using synthetic dsRNAs, termed “small activating RNAs” (saRNAs). It is currently not known whether RNAa is conserved in other organisms.

According to the present invention, the oligonucleotides or “antisense compounds” include antisense oligonucleotides (e.g. RNA, DNA, mimetic, chimera, analog or homolog thereof), ribozymes, external guide sequence (EGS) oligonucleotides, siRNA compounds, single- or double-stranded RNA interference (RNAi) compounds such as siRNA compounds, saRNA, aRNA, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleotide positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines (Hammond et al., (1991) Nat. Rev. Genet., 2, 110-119; Matzke et al., (2001) Curr. Opin. Genet. Dev., 11, 221-227; Sharp, (2001) Genes Dev., 15, 485-490). When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

In one embodiment the invention teaches the combined use of NR2F6 inhibition with other agents which potential killing of endothelial cells associated with pathological angiogenesis. On possible combination agent is rapamycin. In one series of experiments tumor bearing animals where treated with rapamycin and the results showed that CD34(+) blood vessels and LYVE-1(+) lymphatic vessels decreased in the peritumor and intratumor region in rapamycin-treated tumors. Expression of p-4EBP1 and p-S6K1 proteins was downregulated. Expression of both proteins and mRNAs of VEGF-A/VEGFR-2 and VEGF-C/VEGFR-3 was downregulated [405].

### REFERENCES

1. Coley, W. B., The treatment of malignant tumors by repeated inoculations of erysipelas. With a report of ten original cases. 1893. Clin Orthop Relat Res, 1991(262): p. 3-11.
2. Coley, W. B., The Treatment of Sarcoma of the Long Bones. Ann Surg, 1933. 97(3): p. 434-60.
3. McCarthy, E. F., The toxins of William B. Coley and the treatment of bone and soft-tissue sarcomas. Iowa Orthop J, 2006. 26: p. 154-8.
4. Bickels, J., et al., Coley's toxin: historical perspective. Isr Med Assoc J, 2002. 4(6): p. 471-2.
5. Wiemann, B. and C. O. Starnes, Coley's toxins, tumor necrosis factor and cancer research: a historical perspective. Pharmacol Ther, 1994. 64(3): p. 529-64.
6. Starnes, C. O., Coley's toxins. Nature, 1992. 360(6399): p. 23.
7. Brouckaert, P. G., W. Fiers, and F. J. Lejeune, Coley's vaccine and TNF therapy. Nature, 1992. 358(6388): p. 630.
8. Nauts, H. C., G. A. Fowler, and F. H. Bogatko, A review of the influence of bacterial infection and of bacterial products (Coley's toxins) on malignant tumors in man; a critical analysis of 30 inoperable cases treated by Coley's mixed toxins, in which diagnosis was confirmed by microscopic examination selected for special study. Acta Med Scand Suppl, 1953. 276: p. 1-103.
9. Brower, V., Approval of provenge seen as first step for cancer treatment vaccines. J Natl Cancer Inst, 2010. 102(15): p. 1108-10.
10. Lipson, E. J. and C. G. Drake, Ipilimumab: an anti-CTLA-4 antibody for metastatic melanoma. Clin Cancer Res, 2011. 17(22): p. 6958-62.
11. Rajakulendran, T. and D. N. Adam, Spotlight on pembrolizumab in the treatment of advanced melanoma. Drug Des Devel Ther, 2015. 9: p. 2883-6.
12. Raedler, L. A., Opdivo (Nivolumab): Second PD-1 Inhibitor Receives FDA Approval for Unresectable or Metastatic Melanoma. Am Health Drug Benefits, 2015. 8(Spec Feature): p. 180-3.
13. Sweis, R. F. and J. J. Luke, Mechanistic and pharmacologic insights on immune checkpoint inhibitors. Pharmacol Res, 2017. 120: p. 1-9.
14. Martin-Fontecha, A., et al., Induced recruitment of NK cells to lymph nodes provides IFN-gamma for T(H)1 priming. Nat Immunol, 2004. 5(12): p. 1260-5.
15. Morandi, B., et al., NK cells of human secondary lymphoid tissues enhance T cell polarization via IFN-gamma secretion. Eur J Immunol, 2006. 36(9): p. 2394-400.
16. Ksienzyk, A., et al., IRF-I expression is essential for natural killer cells to suppress metastasis. Cancer Res, 2011. 71(20): p. 6410-8.
17. Lopez-Soto, A., et al., Control of Metastasis by NK Cells. Cancer Cell, 2017. 32(2): p. 135-154.
18. Krasnova, Y., et al., Bench to bedside: NK cells and control of metastasis. Clin Immunol, 2017. 177: p. 50-59.
19. Putz, E. M., et al., NK cell heparanase controls tumor invasion and immune surveillance. J Clin Invest, 2017. 127(7): p. 2777-2788.
20. Morvan, M. G. and L. L. Lanier, NK cells and cancer: you can teach innate cells new tricks. Nat Rev Cancer, 2016. 16(1): p. 7-19.
21. Maccalli, C., et al., Soluble NKG2D ligands are biomarkers associated with the clinical outcome to immune checkpoint blockade therapy of metastatic melanoma patients. Oncoimmunology, 2017. 6(7): p. e1323618.
22. Goding, S. R., et al., Adoptive transfer of natural killer cells promotes the anti-tumor efficacy of T cells. Clin Immunol, 2017. 177: p. 76-86.
23. Muraro, E., et al., Improved Natural Killer cell activity and retained anti-tumor CD8(+) T cell responses contribute to the induction of a pathological complete response in HER2-positive breast cancer patients undergoing neoadjuvant chemotherapy. J Transl Med, 2015. 13: p. 204.
24. Lee, S. C., et al., Natural killer (NK): dendritic cell (DC) cross talk induced by therapeutic monoclonal antibody triggers tumor antigen-specific T cell immunity. Immunol Res, 2011. 50(2-3): p. 248-54.
25. Beano, A., et al., Correlation between NK function and response to trastuzumab in metastatic breast cancer patients. J Transl Med, 2008. 6: p. 25.
26. Jaime-Ramirez, A. C., et al., IL-12 enhances the antitumor actions of trastuzumab via NK cell IFN-gamma production. J Immunol, 2011. 186(6): p. 3401-9.
27. Charlebois, R., et al., PolyI.C and CpG Synergize with Anti-ErbB2 mAb for Treatment of Breast Tumors Resistant to Immune Checkpoint Inhibitors. Cancer Res, 2017. 77(2): p. 312-319.
28. Hoshimoto, S., et al., Assessment of prognostic circulating tumor cells in a phase III trial of adjuvant immunotherapy after complete resection of stage IV melanoma. Ann Surg, 2012. 255(2): p. 357-62.
29. Kalani, A. D., et al., Immunotherapy as an adjuvant therapy in the management of advanced, surgically resected, melanoma. G Ital Dermatol Venereol, 2008. 143(1): p. 59-70.
30. Ugel, S., et al., Targeting tumor vasculature: expanding the potential of DNA cancer vaccines. Cancer Immunol Immunother, 2015. 64(10): p. 1339-48.
31. Li, L., et al., Developing a clinical development paradigm for translation of a mammaglobin-A DNA vaccine. Immunotherapy, 2015: p. 1-3.
32. Tiriveedhi, V., et al., Safety and preliminary evidence of biologic efficacy of a mammaglobin-a DNA vaccine in patients with stable metastatic breast cancer. Clin Cancer Res, 2014. 20(23): p. 5964-75.
33. Heller, R. and L. C. Heller, Gene electrotransfer clinical trials. Adv Genet, 2015. 89: p. 235-62.
34. Butterfield, L. H., et al., Alpha fetoprotein DNA prime and adenovirus boost immunization of two hepatocellular cancer patients. J Transl Med, 2014. 12: p. 86.
35. Sharpe, M., et al., Protection of mice from H5N1 influenza challenge by prophylactic DNA vaccination using particle mediated epidermal delivery. Vaccine, 2007. 25(34): p. 6392-8.
36. Loudon, P. T., et al., GM-CSF increases mucosal and systemic immunogenicity of an H1N1 influenza DNA vaccine administered into the epidermis of non-human primates. PLoS One, 2010. 5(6): p. e11021.
37. Jones, S., et al., DNA vaccination protects against an influenza challenge in a double-blind randomised placebo-controlled phase 1b clinical trial. Vaccine, 2009. 27(18): p. 2506-12.
38. Shah, M. A., et al., DNA Mediated Vaccines Delivery Through Nanoparticles. J Nanosci Nanotechnol, 2015. 15(1): p. 41-53.
39. Mpendo, J., et al., A Phase I Double Blind, Placebo-Controlled, Randomized Study of the Safety and Immunogenicity of Electroporated HIV DNA with or without Interleukin 12 in Prime-Boost Combinations with an Ad35 HIV Vaccine in Healthy HIV-Seronegative African Adults. PLoS One, 2015. 10(8): p. e0134287.
40. Keane-Myers, A. M., et al., DNA electroporation of multi-agent vaccines conferring protection against select agent challenge: TriGrid delivery system. Methods Mol Biol, 2014. 1121: p. 325-36.
41. Hooper, J. W., et al., A Phase 1 clinical trial of Hantaan virus and Puumala virus M-segment DNA vaccines for haemorrhagic fever with renal syndrome delivered by intramuscular electroporation. Clin Microbiol Infect, 2014. 20 Suppl 5: p. 110-7.
42. van Furth, R. and Z. A. Cohn, The origin and kinetics of mononuclear phagocytes. J Exp Med, 1968. 128(3): p. 415-35.
43. Wynn, T. A., A. Chawla, and J. W. Pollard, Macrophage biology in development, homeostasis and disease. Nature, 2013. 496(7446): p. 445-55.
44. Smith, T. D., et al., Harnessing macrophage plasticity for tissue regeneration. Adv Drug Deliv Rev, 2017.
45. Vannella, K. M. and T. A. Wynn, Mechanisms of Organ Injury and Repair by Macrophages. Annu Rev Physiol, 2017. 79: p. 593-617.
46. Boddupalli, A., L. Zhu, and K. M. Bratlie, Methods for Implant Acceptance and Wound Healing: Material Selection and Implant Location Modulate Macrophage and Fibroblast Phenotypes. Adv Healthc Mater, 2016. 5(20): p. 2575-2594.
47. Snyder, R. J., et al., Macrophages: A review of their role in wound healing and their therapeutic use. Wound Repair Regen, 2016. 24(4): p. 613-29.
48. Gombozhapova, A., et al., Macrophage activation and polarization in post-infarction cardiac remodeling. J Biomed Sci, 2017. 24(1): p. 13.
49. Hu, Y., et al., Class A scavenger receptor attenuates myocardial infarction-induced cardiomyocyte necrosis through suppressing M1 macrophage subset polarization. Basic Res Cardiol, 2011. 106(6): p. 1311-28.
50. Ma, Y., et al., Matrix metalloproteinase-28 deletion exacerbates cardiac dysfunction and rupture after myocardial infarction in mice by inhibiting M2 macrophage activation. Circ Res, 2013. 112(4): p. 675-88.
51. Lee, C. W., et al., Macrophage heterogeneity of culprit coronary plaques in patients with acute myocardial infarction or stable angina. Am J Clin Pathol, 2013. 139(3): p. 317-22.
52. Yan, X., et al., Temporal dynamics of cardiac immune cell accumulation following acute myocardial infarction. J Mol Cell Cardiol, 2013. 62: p. 24-35.
53. Fernandez-Velasco, M., S. Gonzalez-Ramos, and L. Bosca, Involvement of monocytes/macrophages as key factors in the development and progression of cardiovascular diseases. Biochem J, 2014. 458(2): p. 187-93.
54. de Couto, G., et al., Macrophages mediate cardioprotective cellular post conditioning in acute myocardial infarction. J Clin Invest, 2015. 125(8): p. 3147-62.
55. Guiteras, R., M. Flaquer, and J. M. Cruzado, Macrophage in chronic kidney disease. Clin Kidney J, 2016. 9(6): p. 765-771.
56. Meng, X. M., et al., Macrophage Phenotype in Kidney Injury and Repair. Kidney Dis (Basel), 2015. 1(2): p. 138-46.
57. Yamamoto, S., et al., Atherosclerosis following renal injury is ameliorated by pioglitazone and losartan via macrophage phenotype. Atherosclerosis, 2015. 242(1): p. 56-64.
58. Li, C., et al., Enhanced M1 and Impaired M2 Macrophage Polarization and Reduced Mitochondrial Biogenesis via Inhibition of AMP Kinase in Chronic Kidney Disease. Cell Physiol Biochem, 2015. 36(1): p. 358-72.
59. Sun, Y. Y., et al., Macrophage Phenotype in Liver Injury and Repair. Scand J Immunol, 2017. 85(3): p. 166-174.
60. Gratchev, A., et al., Mphi1 and Mphi2 can be re-polarized by Th2 or Th1 cytokines, respectively, and respond to exogenous danger signals. Immunobiology, 2006. 211(6-8): p. 473-86.
61. Mills, C. D., M1 and M2 Macrophages: Oracles of Health and Disease. Crit Rev Immunol, 2012. 32(6): p. 463-88.
62. Mills, C. D. and K. Ley, M1 and M2 macrophages: the chicken and the egg of immunity. J Innate Immun, 2014. 6(6): p. 716-26.
63. Alsaid, H., et al., Non invasive imaging assessment of the biodistribution of GSK2849330, an ADCC and CDC optimized anti HER3 mAb, and its role in tumor macrophage recruitment in human tumor-bearing mice. PLoS One, 2017. 12(4): p. e0176075.
64. Josephs, D. H., et al., Anti-Folate Receptor-alpha IgE but not IgG Recruits Macrophages to Attack Tumors via TNFalpha/MCP-1 Signaling. Cancer Res, 2017. 77(5): p. 1127-1141.
65. Velmurugan, R., et al., Macrophage-Mediated Trogocytosis Leads to Death of Antibody-Opsonized Tumor Cells. Mol Cancer Ther, 2016. 15(8): p. 1879-89.
66. Gul, N. and M. van Egmond, Antibody-Dependent Phagocytosis of Tumor Cells by Macrophages: A Potent Effector Mechanism of Monoclonal Antibody Therapy of Cancer. Cancer Res, 2015. 75(23): p. 5008-13.
67. Church, A. K., et al., Anti-CD20 monoclonal antibody-dependent phagocytosis of chronic lymphocytic leukaemia cells by autologous macrophages. Clin Exp Immunol, 2016. 183(1): p. 90-101.
68. Shi, Y., et al., Trastuzumab triggers phagocytic killing of high HER2 cancer cells in vitro and in vivo by interaction with Fcgamma receptors on macrophages. J Immunol, 2015. 194(9): p. 4379-86.
69. Weiskopf, K. and I. L. Weissman, Macrophages are critical effectors of antibody therapies for cancer. MAbs, 2015. 7(2): p. 303-10.
70. Oflazoglu, E., et al., Macrophages contribute to the antitumor activity of the anti-CD30 antibody SGN-30. Blood, 2007. 110(13): p. 4370-2.
71. Osman, R., et al., Calreticulin Release at an Early Stage of Death Modulates the Clearance by Macrophages of Apoptotic Cells. Front Immunol, 2017. 8: p. 1034.
72. Feng, M., et al., Macrophages eat cancer cells using their own calreticulin as a guide: roles of TLR and Btk. Proc Natl Acad Sci USA, 2015. 112(7): p. 2145-50.
73. Chao, M. P., et al., Calreticulin is the dominant pro-phagocytic signal on multiple human cancers and is counterbalanced by CD47. Sci Transl Med, 2010. 2(63): p. 63ra94.
74. Murata, Y., et al., The CD47-SIRPalpha signalling system: its physiological roles and therapeutic application. J Biochem, 2014. 155(6): p. 335-44.
75. Roberts, D. D., S. Kaur, and D. R. Soto-Pantoja, Therapeutic targeting of the thrombospondin-1 receptor CD47 to treat liver cancer. J Cell Commun Signal, 2015. 9(1): p. 101-2.
76. Liu, J., et al., Pre-Clinical Development of a Humanized Anti-CD47 Antibody with Anti-Cancer Therapeutic Potential. PLoS One, 2015. 10(9): p. e0137345.
77. Weiskopf, K., et al., CD47-blocking immunotherapies stimulate macrophage-mediated destruction of small-cell lung cancer. J Clin Invest, 2016. 126(7): p. 2610-20.
78. Weiskopf, K., et al., Eradication of Canine Diffuse Large B-Cell Lymphoma in a Murine Xenograft Model with CD47 Blockade and Anti-CD20. Cancer Immunol Res, 2016. 4(12): p. 1072-1087.
79. Zeng, D., et al., A fully human anti-CD47 blocking antibody with therapeutic potential for cancer. Oncotarget, 2016. 7(50): p. 83040-83050.
80. Liljefors, M., et al., Influence of varying doses of granulocyte-macrophage colony-stimulating factor on pharmacokinetics and antibody-dependent cellular cytotoxicity. Cancer Immunol Immunother, 2008. 57(3): p. 379-88.
81. Tarr, P. E., Granulocyte-macrophage colony-stimulating factor and the immune system. Med Oncol, 1996. 13(3): p. 133-40.
82. Ragnhammar, P., et al., Cytotoxicity of white blood cells activated by granulocyte-colony-stimulating factor, granulocyte/macrophage-colony-stimulating factor and macrophage-colony-stimulating factor against tumor cells in the presence of various monoclonal antibodies. Cancer Immunol Immunother, 1994. 39(4): p. 254-62.
83. Ragnhammar, P., Anti-tumoral effect of GM-CSF with or without cytokines and monoclonal antibodies in solid tumors. Med Oncol, 1996. 13(3): p. 167-76.
84. Lin, E. Y., et al., Colony-stimulating factor 1 promotes progression of mammary tumors to malignancy. J Exp Med, 2001. 193(6): p. 727-40.
85. Aharinejad, S., et al., Colony-stimulating factor-1 blockade by antisense oligonucleotides and small interfering RNAs suppresses growth of human mammary tumor xenografts in mice. Cancer Res, 2004. 64(15): p. 5378-84.
86. Lin, E. Y., et al., Macrophages regulate the angiogenic switch in a mouse model of breast cancer. Cancer Res, 2006. 66(23): p. 11238-46.
87. Lin, E. Y. and J. W. Pollard, Tumor-associated macrophages press the angiogenic switch in breast cancer. Cancer Res, 2007. 67(11): p. 5064-6.
88. Zhang, W. J., et al., Hypoxia-inducible factor-1 alpha Correlates with Tumor-Associated Macrophages Infiltration, Influences Survival of Gastric Cancer Patients. J Cancer, 2017. 8(10): p. 1818-1825.
89. Yuan, X., et al., Prognostic significance of tumor-associated macrophages in ovarian cancer: A meta-analysis. Gynecol Oncol, 2017. 147(1): p. 181-187.
90. Ma, C., et al., CD163-positive cancer cells are potentially associated with high malignant potential in clear cell renal cell carcinoma. Med Mol Morphol, 2017.
91. Shi, Y., et al., Tumour-associated macrophages secrete pleiotrophin to promote PTPRZ1 signalling in glioblastoma stem cells for tumour growth. Nat Commun, 2017. 8: p. 15080.
92. Zhao, X., et al., Prognostic significance of tumor-associated macrophages in breast cancer: a meta-analysis of the literature. Oncotarget, 2017. 8(18): p. 30576-30586.
93. Pearce, O. M., et al., Inverse hormesis of cancer growth mediated by narrow ranges of tumor-directed antibodies. Proc Natl Acad Sci USA, 2014. 111(16): p. 5998-6003.
94. Pander, J., et al., Activation of tumor-promoting type 2 macrophages by EGFR-targeting antibody cetuximab. Clin Cancer Res, 2011. 17(17): p. 5668-73.
95. Clynes, R. A., et al., Inhibitory Fc receptors modulate in vivo cytotoxicity against tumor targets. Nat Med, 2000. 6(4): p. 443-6.
96. Pricop, L., et al., Differential modulation of stimulatory and inhibitory Fc gamma receptors on human monocytes by Th1 and Th2 cytokines. J Immunol, 2001. 166(1): p. 531-7.
97. Tridandapani, S., et al., Regulated expression and inhibitory function of Fcgamma RIIb in human monocytic cells. J Biol Chem, 2002. 277(7): p. 5082-9.
98. Joshi, T., et al., Molecular analysis of expression and function of hFcgammaRllbl and b2 isoforms in myeloid cells. Mol Immunol, 2006. 43(7): p. 839-50.
99. Wijngaarden, S., et al., A shift in the balance of inhibitory and activating Fcgamma receptors on monocytes toward the inhibitory Fcgamma receptor IIb is associated with prevention of monocyte activation in rheumatoid arthritis. Arthritis Rheum, 2004. 50(12): p. 3878-87.
100. Butchar, J. P., et al., Reciprocal regulation of activating and inhibitory Fc{gamma} receptors by TLR7/8 activation: implications for tumor immunotherapy. Clin Cancer Res, 2010. 16(7): p. 2065-75.
101. Fatehchand, K., et al., Toll-like Receptor 4 Ligands Down-regulate Fcgamma Receptor IIb (FcgammaRIIb) via MARCH3 Protein-mediated Ubiquitination. J Biol Chem, 2016. 291(8): p. 3895-904.
102. Ghochikyan, A., et al., Targeting TLR-4 with a novel pharmaceutical grade plant derived agonist, Immunomax(R), as a therapeutic strategy for metastatic breast cancer. J Transl Med, 2014. 12: p. 322.
103. Oronsky, B., et al., RRx-001: a systemically non-toxic M2-to-M1 macrophage stimulating and prosensitizing agent in Phase II clinical trials. Expert Opin Investig Drugs, 2017. 26(1): p. 109-119.
104. Lee, C., et al., Melittin suppresses tumor progression by regulating tumor-associated macrophages in a Lewis lung carcinoma mouse model. Oncotarget, 2017. 8(33): p. 54951-54965.
105. Zhang, Q., et al., Clinical Effects of CpG-Based Treatment on the Efficacy of Hepatocellular Carcinoma by Skewing Polarization Toward M1 Macrophage from M2. Cancer Biother Radiopharm, 2017. 32(6): p. 215-219.
106. Sato, T., et al., Intrapulmonary Delivery of CpG Microparticles Eliminates Lung Tumors. Mol Cancer Ther, 2015. 14(10): p. 2198-205.
107. Chiang, C. F., et al., Metformin-treated cancer cells modulate macrophage polarization through AMPK-NF-kappaB signaling. Oncotarget, 2017. 8(13): p. 20706-20718.
108. Kang, H., et al., Puerarin inhibits M2 polarization and metastasis of tumor-associated macrophages from NSCLC xenograft model via inactivating MEK/ERK 1/2 pathway. Int J Oncol, 2017. 50(2): p. 545-554.
109. Jia, X., et al., Emodin suppresses pulmonary metastasis of breast cancer accompanied with decreased macrophage recruitment and M2 polarization in the lungs. Breast Cancer Res Treat, 2014. 148(2): p. 291-302.
110. Xue, N., et al., Chlorogenic acid inhibits glioblastoma growth through repolarizating macrophage from M2 to M1 phenotype. Sci Rep, 2017. 7: p. 39011.
111. Sloan, E. K., et al., The sympathetic nervous system induces a metastatic switch in primary breast cancer. Cancer Res, 2010. 70(18): p. 7042-52.
112. Liu, B., et al., Polarization of M1 tumor associated macrophage promoted by the activation of TLR3 signal pathway. Asian Pac J Trop Med, 2016. 9(5): p. 484-8.
113. Liu, Q., et al., NMAAP1 Expressed in BCG-Activated Macrophage Promotes M1 Macrophage Polarization. Mol Cells, 2015. 38(10): p. 886-94.
114. Liu, Y., et al., Polysaccharide Agaricus blazei Murill stimulates myeloid derived suppressor cell differentiation from M2 to M1 type, which mediates inhibition of tumour immune-evasion via the Toll-like receptor 2 pathway. Immunology, 2015. 146(3): p. 379-91.
115. Yang, Y., et al., LPS converts Gr-1(+)CD115(+) myeloid-derived suppressor cells from M2 to M1 via P38 MAPK. Exp Cell Res, 2013. 319(12): p. 1774-83.
116. Sanchez-Quesada, C., A. Lopez-Biedma, and J. J. Gaforio, Maslinic Acid enhances signals for the recruitment of macrophages and their differentiation to m1 state. Evid Based Complement Alternat Med, 2015. 2015: p. 654721.
117. Dominguez-Soto, A., et al., Intravenous immunoglobulin promotes antitumor responses by modulating macrophage polarization. J Immunol, 2014. 193(10): p. 5181-9.
118. Yin, Y., et al., Phosphatidylserine-targeting antibody induces M1 macrophage polarization and promotes myeloid-derived suppressor cell differentiation. Cancer Immunol Res, 2013. 1(4): p. 256-68.
119. Deng, R., et al., Dimethyl Sulfoxide Suppresses Mouse 4T1 Breast Cancer Growth by Modulating Tumor-Associated Macrophage Differentiation. J Breast Cancer, 2014. 17(1): p. 25-32.
120. Mitsuhashi, A., et al., Surfactant protein A suppresses lung cancer progression by regulating the polarization of tumor-associated macrophages. Am J Pathol, 2013. 182(5): p. 1843-53.
121. Coscia, M., et al., Zoledronic acid repolarizes tumour-associated macrophages and inhibits mammary carcinogenesis by targeting the mevalonate pathway. J Cell Mol Med, 2010. 14(12): p. 2803-15.
122. Eriksson, F., et al., Tumor-specific bacteriophages induce tumor destruction through activation of tumor-associated macrophages. J Immunol, 2009. 182(5): p. 3105-11.
123. Steinman, R. M. and Z. A. Cohn, Identification of a novel cell type in peripheral lymphoid organs of mice. I. Morphology, quantitation, tissue distribution. J Exp Med, 1973. 137(5): p. 1142-62.
124. Banchereau, J. and R. M. Steinman, Dendritic cells and the control of immunity. Nature, 1998. 392(6673): p. 245-52.
125. Trombetta, E. S. and I. Mellman, Cell biology of antigen processing in vitro and in vivo. Annu Rev Immunol, 2005. 23: p. 975-1028.
126. Itano, A. A. and M. K. Jenkins, Antigen presentation to naive CD4 T cells in the lymph node. Nat Immunol, 2003. 4(8): p. 733-9.
127. Tjoa, B. A., et al., Evaluation of phase I/II clinical trials in prostate cancer with dendritic cells and PSMA peptides. Prostate, 1998. 36(1): p. 39-44.
128. Murphy, G. P., et al., Infusion of dendritic cells pulsed with HLA-A2-specific prostate-specific membrane antigen peptides: a phase II prostate cancer vaccine trial involving patients with hormone-refractory metastatic disease. Prostate, 1999. 38(1): p. 73-8.
129. Lodge, P. A., et al., Dendritic cell-based immunotherapy of prostate cancer: immune monitoring of a phase II clinical trial. Cancer Res, 2000. 60(4): p. 829-33.
130. Burch, P. A., et al., Priming tissue-specific cellular immunity in a phase I trial of autologous dendritic cells for prostate cancer. Clin Cancer Res, 2000. 6(6): p. 2175-82.
131. Nestle, F. O., et al., Vaccination of melanoma patients with peptide- or tumor lysate-pulsed dendritic cells. Nat Med, 1998. 4(3): p. 328-32.
132. Chakraborty, N. G., et al., Immunization with a tumor-cell-lysate-loaded autologous-antigen-presenting-cell-based vaccine in melanoma. Cancer Immunol Immunother, 1998. 47(1): p. 58-64.
133. Wang, F., et al., Phase I trial of a MART-1 peptide vaccine with incomplete Freund's adjuvantfor resected high-risk melanoma. Clin Cancer Res, 1999. 5(10): p. 2756-65.
134. Thurner, B., et al., Vaccination with mage-3A1 peptide-pulsed mature, monocyte-derived dendritic cells expands specific cytotoxic T cells and induces regression of some metastases in advanced stage IV melanoma. J Exp Med, 1999. 190(11): p. 1669-78.
135. Thomas, R., et al., Immature human monocyte-derived dendritic cells migrate rapidly to draining lymph nodes after intradermal injection for melanoma immunotherapy. Melanoma Res, 1999. 9(5): p. 474-81.
136. Mackensen, A., et al., Phase I study in melanoma patients of a vaccine with peptide-pulsed dendritic cells generated in vitro from CD34(+) hematopoietic progenitor cells. Int J Cancer, 2000. 86(3): p. 385-92.
137. Panelli, M. C., et al., Phase 1 study inpatients with metastatic melanoma of immunization with dendritic cells presenting epitopes derived from the melanoma-associated antigens MART-1 and gp100. J Immunother, 2000. 23(4): p. 487-98.
138. Schuler-Thurner, B., et al., Mage-3 and influenza-matrix peptide-specific cytotoxic T cells are inducible in terminal stage HLA-A2.1+ melanoma patients by mature monocyte-derived dendritic cells. J Immunol, 2000. 165(6): p. 3492-6.
139. Lau, R., et al., Phase I trial of intravenous peptide-pulsed dendritic cells in patients with metastatic melanoma. J Immunother, 2001. 24(1): p. 66-78.
140. Banchereau, J., et al., Immune and clinical responses in patients with metastatic melanoma to CD34(+) progenitor-derived dendritic cell vaccine. Cancer Res, 2001. 61(17): p. 6451-8.
141. Schuler-Thurner, B., et al., Rapid induction of tumor-specific type 1 T helper cells in metastatic melanoma patients by vaccination with mature, cryopreserved, peptide-loaded monocyte-derived dendritic cells. J Exp Med, 2002. 195(10): p. 1279-88.
142. Palucka, A. K., et al., Single injection of CD34+ progenitor-derived dendritic cell vaccine can lead to induction of T-cell immunity in patients with stage IV melanoma. J Immunother, 2003. 26(5): p. 432-9.
143. Bedrosian, I., et al., Intranodal administration of peptide-pulsed mature dendritic cell vaccines results in superior CD8+T-cell function in melanoma patients. J Clin Oncol, 2003. 21(20): p. 3826-35.
144. Slingluff, C. L., Jr., et al., Clinical and immunologic results of a randomized phase II trial of vaccination using four melanoma peptides either administered in granulocyte-macrophage colony-stimulating factor in adjuvant or pulsed on dendritic cells. J Clin Oncol, 2003. 21(21): p. 4016-26.
145. Hersey, P., et al., Phase I/II study of treatment with dendritic cell vaccines in patients with disseminated melanoma. Cancer Immunol Immunother, 2004. 53(2): p. 125-34.
146. Vilella, R., et al., Pilot study of treatment of biochemotherapy-refractory stage IV melanoma patients with autologous dendritic cells pulsed with a heterologous melanoma cell line lysate. Cancer Immunol Immunother, 2004. 53(7): p. 651-8.
147. Palucka, A. K., et al., Spontaneous proliferation and type 2 cytokine secretion by CD4+T cells in patients with metastatic melanoma vaccinated with antigen-pulsed dendritic cells. J Clin Immunol, 2005. 25(3): p. 288-95.
148. Banchereau, J., et al., Immune and clinical outcomes in patients with stage IV melanoma vaccinated with peptide-pulsed dendritic cells derived from CD34+ progenitors and activated with type I interferon. J Immunother, 2005. 28(5): p. 505-16.
149. Trakatelli, M., et al., A new dendritic cell vaccine generated with interleukin-3 and interferon-beta induces CD8+ T cell responses against NA17-A2 tumor peptide in melanoma patients. Cancer Immunol Immunother, 2006. 55(4): p. 469-74.
150. Salcedo, M., et al., Vaccination of melanoma patients using dendritic cells loaded with an allogeneic tumor cell lysate. Cancer Immunol Immunother, 2006. 55(7): p. 819-29.
151. Linette, G. P., et al., Immunization using autologous dendritic cells pulsed with the melanoma-associated antigen gp100-derived G280-9V peptide elicits CD8+ immunity. Clin Cancer Res, 2005. 11(21): p. 7692-9.
152. Escobar, A., et al., Dendritic cell immunizations alone or combined with low doses of interleukin-2 induce specific immune responses in melanoma patients. Clin Exp Immunol, 2005. 142(3): p. 555-68.
153. Tuettenberg, A., et al., Induction of strong and persistent MelanA/MART-1-specific immune responses by adjuvant dendritic cell-based vaccination of stage II melanoma patients. Int J Cancer, 2006. 118(10): p. 2617-27.
154. Schadendorf, D., et al., Dacarbazine (DTIC) versus vaccination with autologous peptide-pulsed dendritic cells (DC) in first-line treatment of patients with metastatic melanoma: a randomized phase III trial of the DC study group of the DeCOG. Ann Oncol, 2006. 17(4): p. 563-70.
155. Di Pucchio, T., et al., Immunization of stage IV melanoma patients with Melan-A/MART-1 and gp100 peptides plus IFN-alpha results in the activation of specific CD8(+) T cells and monocyte/dendritic cell precursors. Cancer Res, 2006. 66(9): p. 4943-51.
156. Nakai, N., et al., Vaccination of Japanese patients with advanced melanoma with peptide, tumor lysate or both peptide and tumor lysate-pulsed mature, monocyte-derived dendritic cells. J Dermatol, 2006. 33(7): p. 462-72.
157. Palucka, A. K., et al., Dendritic cells loaded with killed allogeneic melanoma cells can induce objective clinical responses and MART-1 specific CD8+T-cell immunity. J Immunother, 2006. 29(5): p. 545-57.
158. Lesimple, T., et al., Immunologic and clinical effects of injecting mature peptide-loaded dendritic cells by intralymphatic and intranodal routes in metastatic melanoma patients. Clin Cancer Res, 2006. 12(24): p. 7380-8.
159. Guo, J., et al., Intratumoral injection of dendritic cells in combination with local hyperthermia induces systemic antitumor effect in patients with advanced melanoma. Int J Cancer, 2007. 120(11): p. 2418-25.
160. O'Rourke, M. G., et al., Dendritic cell immunotherapy for stage IV melanoma. Melanoma Res, 2007. 17(5): p. 316-22.
161. Bercovici, N., et al., Analysis and characterization of antitumor T-cell response after administration of dendritic cells loaded with allogeneic tumor lysate to metastatic melanoma patients. J Immunother, 2008. 31(1): p. 101-12.
162. Hersey, P., et al., Phase I/II study of treatment with matured dendritic cells with or without low dose IL-2 in patients with disseminated melanoma. Cancer Immunol Immunother, 2008. 57(7): p. 1039-51.
163. von Euw, E. M., et al., A phase I clinical study of vaccination of melanoma patients with dendritic cells loaded with allogeneic apoptotic/necrotic melanoma cells. Analysis of toxicity and immune response to the vaccine and of IL-10-1082 promoter genotype as predictor of disease progression. J Transl Med, 2008. 6: p. 6.
164. Carrasco, J., et al., Vaccination of a melanoma patient with mature dendritic cells pulsed with MAGE-3 peptides triggers the activity of nonvaccine anti-tumor cells. J Immunol, 2008. 180(5): p. 3585-93.
165. Redman, B. G., et al., Phase Ib trial assessing autologous, tumor-pulsed dendritic cells as a vaccine administered with or without IL-2 in patients with metastatic melanoma. J Immunother, 2008. 31(6): p. 591-8.
166. Daud, A. I., et al., Phenotypic and functional analysis of dendritic cells and clinical outcome in patients with high-risk melanoma treated with adjuvant granulocyte macrophage colony-stimulating factor. J Clin Oncol, 2008. 26(19): p. 3235-41.
167. Engell-Noerregaard, L., et al., Review of clinical studies on dendritic cell-based vaccination of patients with malignant melanoma: assessment of correlation between clinical response and vaccine parameters. Cancer Immunol Immunother, 2009. 58(1): p. 1-14.
168. Nakai, N., et al., Immunohistological analysis of peptide-induced delayed-type hypersensitivity in advanced melanoma patients treated with melanoma antigen-pulsed mature monocyte-derived dendritic cell vaccination. J Dermatol Sci, 2009. 53(1): p. 40-7.
169. Dillman, R. O., et al., Phase II trial of dendritic cells loaded with antigens from self-renewing, proliferating autologous tumor cells as patient-specific antitumor vaccines in patients with metastatic melanoma: final report. Cancer Biother Radiopharm, 2009. 24(3): p. 311-9.
170. Chang, J. W., et al., Immunotherapy with dendritic cells pulsed by autologous dactinomycin-induced melanoma apoptotic bodies for patients with malignant melanoma. Melanoma Res, 2009. 19(5): p. 309-15.
171. Trepiakas, R., et al., Vaccination with autologous dendritic cells pulsed with multiple tumor antigens for treatment of patients with malignant melanoma: results from a phase I/II trial. Cytotherapy, 2010. 12(6): p. 721-34.
172. Jacobs, J. F., et al., Dendritic cell vaccination in combination with anti-CD25 monoclonal antibody treatment: a phase I/II study in metastatic melanoma patients. Clin Cancer Res, 2010. 16(20): p. 5067-78.
173. Ribas, A., et al., Multicenter phase II study of matured dendritic cells pulsed with melanoma cell line lysates in patients with advanced melanoma. J Transl Med, 2010. 8: p. 89.
174. Ridolfi, L., et al., Unexpected high response rate to traditional therapy after dendritic cell-based vaccine in advanced melanoma: update of clinical outcome and subgroup analysis. Clin Dev Immunol, 2010. 2010: p. 504979.
175. Cornforth, A. N., et al., Resistance to the proapoptotic effects of interferon-gamma on melanoma cells used in patient-specific dendritic cell immunotherapy is associated with improved overall survival. Cancer Immunol Immunother, 2011. 60(1): p. 123-31.
176. Lesterhuis, W. J., et al., Wild-type and modified gp100 peptide-pulsed dendritic cell vaccination of advanced melanoma patients can lead to long-term clinical responses independent of the peptide used. Cancer Immunol Immunother, 2011. 60(2): p. 249-60.
177. Bjoern, J., et al., Changes in peripheral blood level of regulatory T cells in patients with malignant melanoma during treatment with dendritic cell vaccination and low-dose IL-2. Scand J Immunol, 2011. 73(3): p. 222-33.
178. Steele, J. C., et al., Phase I/II trial of a dendritic cell vaccine transfected with DNA encoding melan A and gp100 for patients with metastatic melanoma. Gene Ther, 2011. 18(6): p. 584-93.
179. Kim, D. S., et al., Immunotherapy of malignant melanoma with tumor lysate-pulsed autologous monocyte-derived dendritic cells. Yonsei Med J, 2011. 52(6): p. 990-8.
180. Ellebaek, E., et al., Metastatic melanoma patients treated with dendritic cell vaccination, Interleukin-2 and metronomic cyclophosphamide: results from a phase II trial. Cancer Immunol Immunother, 2012. 61(10): p. 1791-804.
181. Dillman, R. O., et al., Tumor stem cell antigens as consolidative active specific immunotherapy: a randomized phase II trial of dendritic cells versus tumor cells in patients with metastatic melanoma. J Immunother, 2012. 35(8): p. 641-9.
182. Dannull, J., et al., Melanoma immunotherapy using mature DCs expressing the constitutive proteasome. J Clin Invest, 2013. 123(7): p. 3135-45.
183. Finkelstein, S. E., et al., Combination of external beam radiotherapy (EBRT) with intratumoral injection of dendritic cells as neo-adjuvant treatment of high-risk soft tissue sarcoma patients. Int J Radiat Oncol Biol Phys, 2012. 82(2): p. 924-32.
184. Stift, A., et al., Dendritic cell vaccination in medullary thyroid carcinoma. Clin Cancer Res, 2004. 10(9): p. 2944-53.
185. Kuwabara, K., et al., Results of a phase I clinical study using dendritic cell vaccinations for thyroid cancer. Thyroid, 2007. 17(1): p. 53-8.
186. Bachleitner-Hofmann, T., et al., Pilot trial of autologous dendritic cells loaded with tumor lysate(s) from allogeneic tumor cell lines in patients with metastatic medullary thyroid carcinoma. Oncol Rep, 2009. 21(6): p. 1585-92.
187. Yu, J. S., et al., Vaccination of malignant glioma patients with peptide-pulsed dendritic cells elicits systemic cytotoxicity and intracranial T-cell infiltration. Cancer Res, 2001. 61(3): p. 842-7.
188. Yamanaka, R., et al., Vaccination of recurrent glioma patients with tumour lysate-pulsed dendritic cells elicits immune responses: results of a clinical phase I/II trial. Br J Cancer, 2003. 89(7): p. 1172-9.
189. Yu, J. S., et al., Vaccination with tumor lysate-pulsed dendritic cells elicits antigen-specific, cytotoxic T-cells in patients with malignant glioma. Cancer Res, 2004. 64(14): p. 4973-9.
190. Yamanaka, R., et al., Tumor lysate and IL-18 loaded dendritic cells elicits Th1 response, tumor-specific CD8+ cytotoxic T cells in patients with malignant glioma. J Neurooncol, 2005. 72(2): p. 107-13.
191. Yamanaka, R., et al., Clinical evaluation of dendritic cell vaccination for patients with recurrent glioma: results of a clinical phase I/II trial. Clin Cancer Res, 2005. 11(11): p. 4160-7.
192. Liau, L. M., et al., Dendritic cell vaccination in glioblastoma patients induces systemic and intracranial T-cell responses modulated by the local central nervous system tumor microenvironment. Clin Cancer Res, 2005. 11(15): p. 5515-25.
193. Walker, D. G., et al., Results of a phase I dendritic cell vaccine trial for malignant astrocytoma: potential interaction with adjuvant chemotherapy. J Clin Neurosci, 2008. 15(2): p. 114-21.
194. Leplina, O. Y., et al., Use of interferon-alpha-induced dendritic cells in the therapy of patients with malignant brain gliomas. Bull Exp Biol Med, 2007. 143(4): p. 528-34.
195. De Vleeschouwer, S., et al., Postoperative adjuvant dendritic cell-based immunotherapy in patients with relapsed glioblastoma multiforme. Clin Cancer Res, 2008. 14(10): p. 3098-104.
196. Ardon, H., et al., Adjuvant dendritic cell-based tumour vaccination for children with malignant brain tumours. Pediatr Blood Cancer, 2010. 54(4): p. 519-25.
197. Prins, R. M., et al., Gene expression profile correlates with T-cell infiltration and relative survival in glioblastoma patients vaccinated with dendritic cell immunotherapy. Clin Cancer Res, 2011. 17(6): p. 1603-15.
198. Okada, H., et al., Induction of CD8+T-cell responses against novel glioma-associated antigen peptides and clinical activity by vaccinations with [alpha]-type 1 polarized dendritic cells and polyinosinic-polycytidylic acid stabilized by lysine and carboxymethylcellulose in patients with recurrent malignant glioma. J Clin Oncol, 2011. 29(3): p. 330-6.
199. Fadul, C. E., et al., Immune response in patients with newly diagnosed glioblastoma multiforme treated with intranodal autologous tumor lysate-dendritic cell vaccination after radiation chemotherapy. J Immunother, 2011. 34(4): p. 382-9.
200. Chang, C. N., et al., A phase I/II clinical trial investigating the adverse and therapeutic effects of a postoperative autologous dendritic cell tumor vaccine in patients with malignant glioma. J Clin Neurosci, 2011. 18(8): p. 1048-54.
201. Cho, D. Y., et al., Adjuvant immunotherapy with whole-cell lysate dendritic cells vaccine for glioblastoma multiforme: a phase II clinical trial. World Neurosurg, 2012. 77(5-6): p. 736-44.
202. Iwami, K., et al., Peptide-pulsed dendritic cell vaccination targeting interleukin-13 receptor alpha2 chain in recurrent malignant glioma patients with HLA-A*24/A*02 allele. Cytotherapy, 2012. 14(6): p. 733-42.
203. Fong, B., et al., Monitoring of regulatory T cell frequencies and expression of CTLA-4 on T cells, before and after DC vaccination, can predict survival in GBM patients. PLoS One, 2012. 7(4): p. e32614.
204. De Vleeschouwer, S., et al., Stratification according to HGG-IMMUNO RPA model predicts outcome in a large group of patients with relapsed malignant glioma treated by adjuvant postoperative dendritic cell vaccination. Cancer Immunol Immunother, 2012. 61(11): p. 2105-12.
205. Phuphanich, S., et al., Phase I trial of a multi-epitope-pulsed dendritic cell vaccine for patients with newly diagnosed glioblastoma. Cancer Immunol Immunother, 2013. 62(1): p. 125-35.
206. Akiyama, Y., et al., alpha-type-1 polarized dendritic cell-based vaccination in recurrent high-grade glioma: a phase I clinical trial. BMC Cancer, 2012. 12: p. 623.
207. Prins, R. M., et al., Comparison of glioma-associated antigen peptide-loaded versus autologous tumor lysate-loaded dendritic cell vaccination in malignant glioma patients. J Immunother, 2013. 36(2): p. 152-7.
208. Shah, A. H., et al., Dendritic cell vaccine for recurrent high-grade gliomas in pediatric and adult subjects: clinical trial protocol. Neurosurgery, 2013. 73(5): p. 863-7.
209. Reichardt, V. L., et al., Idiotype vaccination using dendritic cells after autologous peripheral blood stem cell transplantation for multiple myeloma—a feasibility study. Blood, 1999. 93(7): p. 2411-9.
210. Lim, S. H. and R. Bailey-Wood, Idiotypic protein-pulsed dendritic cell vaccination in multiple myeloma. Int J Cancer, 1999. 83(2): p. 215-22.
211. Motta, M. R., et al., Generation of dendritic cells from CD14+ monocytes positively selected by immunomagnetic adsorption for multiple myeloma patients enrolled in a clinical trial of anti-idiotype vaccination. Br J Haematol, 2003. 121(2): p. 240-50.
212. Reichardt, V. L., et al., Idiotype vaccination of multiple myeloma patients using monocyte-derived dendritic cells. Haematologica, 2003. 88(10): p. 1139-49.
213. Guardino, A. E., et al., Production of myeloid dendritic cells (DC) pulsed with tumor-specific idiotype protein for vaccination of patients with multiple myeloma. Cytotherapy, 2006. 8(3): p. 277-89.
214. Lacy, M. Q., et al., Idiotype-pulsed antigen-presenting cells following autologous transplantation for multiple myeloma may be associated with prolonged survival. Am J Hematol, 2009. 84(12): p. 799-802.
215. Yi, Q., et al., Optimizing dendritic cell-based immunotherapy in multiple myeloma: intranodal injections of idiotype-pulsed CD40 ligand-matured vaccines led to induction of type-1 and cytotoxic T-cell immune responses in patients. Br J Haematol, 2010. 150(5): p. 554-64.
216. Rollig, C., et al., Induction of cellular immune responses in patients with stage-I multiple myeloma after vaccination with autologous idiotype-pulsed dendritic cells. J Immunother, 2011. 34(1): p. 100-6.
217. Zahradova, L., et al., Efficacy and safety of Id-protein-loaded dendritic cell vaccine in patients with multiple myeloma—phase II study results. Neoplasma, 2012. 59(4): p. 440-9.
218. Timmerman, J. M., et al., Idiotype-pulsed dendritic cell vaccination for B-cell lymphoma: clinical and immune responses in 35 patients. Blood, 2002. 99(5): p. 1517-26.
219. Maier, T., et al., Vaccination of patients with cutaneous T-cell lymphoma using intranodal injection of autologous tumor-lysate-pulsed dendritic cells. Blood, 2003. 102(7): p. 2338-44.
220. Di Nicola, M., et al., Vaccination with autologous tumor-loaded dendritic cells induces clinical and immunologic responses in indolent B-cell lymphoma patients with relapsed and measurable disease: a pilot study. Blood, 2009. 113(1): p. 18-27.
221. Hus, I., et al., Allogeneic dendritic cells pulsed with tumor lysates or apoptotic bodies as immunotherapy for patients with early-stage B-cell chronic lymphocytic leukemia. Leukemia, 2005. 19(9): p. 1621-7.
222. Li, L., et al., Immunotherapy for patients with acute myeloid leukemia using autologous dendritic cells generated from leukemic blasts. Int J Oncol, 2006. 28(4): p. 855-61.
223. Roddie, H., et al., Phase I/II study of vaccination with dendritic-like leukaemia cells for the immunotherapy of acute myeloid leukaemia. Br J Haematol, 2006. 133(2): p. 152-7.
224. Litzow, M. R., et al., Testing the safety of clinical-grade mature autologous myeloid DC in a phase I clinical immunotherapy trial of CML. Cytotherapy, 2006. 8(3): p. 290-8.
225. Westermann, J., et al., Vaccination with autologous non-irradiated dendritic cells in patients with bcr/abl+ chronic myeloid leukaemia. Br J Haematol, 2007. 137(4): p. 297-306.
226. Hus, I., et al., Vaccination of B-CLL patients with autologous dendritic cells can change the frequency of leukemia antigen-specific CD8+ T cells as well as CD4+CD25+FoxP3+ regulatory T cells toward an antileukemia response. Leukemia, 2008. 22(5): p. 1007-17.
227. Palma, M., et al., Development of a dendritic cell-based vaccine for chronic lymphocytic leukemia. Cancer Immunol Immunother, 2008. 57(11): p. 1705-10.
228. Van Tendeloo, V. F., et al., Induction of complete and molecular remissions in acute myeloid leukemia by Wilms' tumor 1 antigen-targeted dendritic cell vaccination. Proc Natl Acad Sci USA, 2010. 107(31): p. 13824-9.
229. Iwashita, Y., et al., A phase I study of autologous dendritic cell-based immunotherapy for patients with unresectable primary liver cancer. Cancer Immunol Immunother, 2003. 52(3): p. 155-61.
230. Lee, W. C., et al., Vaccination of advanced hepatocellular carcinoma patients with tumor lysate-pulsed dendritic cells: a clinical trial. J Immunother, 2005. 28(5): p. 496-504.
231. Butterfield, L. H., et al., A phase I/II trial testing immunization of hepatocellular carcinoma patients with dendritic cells pulsed with four alpha-fetoprotein peptides. Clin Cancer Res, 2006. 12(9): p. 2817-25.
232. Palmer, D. H., et al., A phase II study of adoptive immunotherapy using dendritic cells pulsed with tumor lysate in patients with hepatocellular carcinoma. Hepatology, 2009. 49(1): p. 124-32.
233. El Ansary, M., et al., Immunotherapy by autologous dendritic cell vaccine in patients with advanced HCC. J Cancer Res Clin Oncol, 2013. 139(1): p. 39-48.
234. Tada, F., et al., Phase I/II study of immunotherapy using tumor antigen-pulsed dendritic cells in patients with hepatocellular carcinoma. Int J Oncol, 2012. 41(5): p. 1601-9.
235. Ueda, Y., et al., Dendritic cell-based immunotherapy of cancer with carcinoembryonic antigen-derived, HLA-A24-restricted CTL epitope: Clinical outcomes of 18 patients with metastatic gastrointestinal or lung adenocarcinomas. Int J Oncol, 2004. 24(4): p. 909-17.
236. Hirschowitz, E. A., et al., Autologous dendritic cell vaccines for non-small-cell lung cancer. J Clin Oncol, 2004. 22(14): p. 2808-15.
237. Chang, G. C., et al., A pilot clinical trial of vaccination with dendritic cells pulsed with autologous tumor cells derived from malignant pleural effusion in patients with late-stage lung carcinoma. Cancer, 2005. 103(4): p. 763-71.
238. Yannelli, J. R., et al., The large scale generation of dendritic cells for the immunization of patients with non-small cell lung cancer (NSCLC). Lung Cancer, 2005. 47(3): p. 337-50.
239. Ishikawa, A., et al., A phase I study of alpha-galactosylceramide (KRN7000)-pulsed dendritic cells in patients with advanced and recurrent non-small cell lung cancer. Clin Cancer Res, 2005. 11(5): p. 1910-7.
240. Antonia, S. J., et al., Combination of p53 cancer vaccine with chemotherapy in patients with extensive stage small cell lung cancer. Clin Cancer Res, 2006. 12(3 Pt 1): p. 878-87.
241. Perrot, I., et al., Dendritic cells infiltrating human non-small cell lung cancer are blocked at immature stage. J Immunol, 2007. 178(5): p. 2763-9.
242. Hirschowitz, E. A., et al., Immunization of NSCLC patients with antigen-pulsed immature autologous dendritic cells. Lung Cancer, 2007. 57(3): p. 365-72.
243. Baratelli, F., et al., Pre-clinical characterization of GMP grade CCL21-gene modified dendritic cells for application in a phase I trial in non-small cell lung cancer. J Transl Med, 2008. 6: p. 38.
244. Hegmans, J. P., et al., Consolidative dendritic cell-based immunotherapy elicits cytotoxicity against malignant mesothelioma. Am J Respir Crit Care Med, 2010. 181(12): p. 1383-90.
245. Um, S. J., et al., Phase I study of autologous dendritic cell tumor vaccine in patients with non-small cell lung cancer. Lung Cancer, 2010. 70(2): p. 188-94.
246. Chiappori, A. A., et al., INGN-225: a dendritic cell-based p53 vaccine (Ad.p53-DC) in small cell lung cancer: observed association between immune response and enhanced chemotherapy effect. Expert Opin Biol Ther, 2010. 10(6): p. 983-91.
247. Perroud, M. W., Jr., et al., Mature autologous dendritic cell vaccines in advanced non-small cell lung cancer: a phase I pilot study. J Exp Clin Cancer Res, 2011. 30: p. 65.
248. Skachkova, O. V., et al., Immunological markers of anti-tumor dendritic cells vaccine efficiency in patients with non-small cell lung cancer. Exp Oncol, 2013. 35(2): p. 109-13.
249. Hernando, J. J., et al., Vaccination with autologous tumour antigen-pulsed dendritic cells in advanced gynaecological malignancies: clinical and immunological evaluation of a phase I trial. Cancer Immunol Immunother, 2002. 51(1): p. 45-52.
250. Rahma, O. E., et al., A gynecologic oncology group phase II trial of two p53 peptide vaccine approaches: subcutaneous injection and intravenous pulsed dendritic cells in high recurrence risk ovarian cancer patients. Cancer Immunol Immunother, 2012. 61(3): p. 373-84.
251. Chu, C. S., et al., Phase I/II randomized trial of dendritic cell vaccination with or without cyclophosphamide for consolidation therapy of advanced ovarian cancer in first or second remission. Cancer Immunol Immunother, 2012. 61(5): p. 629-41.
252. Kandalaft, L. E., et al., A Phase I vaccine trial using dendritic cells pulsed with autologous oxidized lysate for recurrent ovarian cancer. J Transl Med, 2013. 11: p. 149.
253. Lepisto, A. J., et al., A phase I/II study of a MUC1 peptide pulsed autologous dendritic cell vaccine as adjuvant therapy inpatients with resected pancreatic and biliary tumors. Cancer Ther, 2008. 6(B): p. 955-964.
254. Rong, Y., et al., A phase I pilot trial of MUC1-peptide-pulsed dendritic cells in the treatment of advanced pancreatic cancer. Clin Exp Med, 2012. 12(3): p. 173-80.
255. Endo, H., et al., Phase I trial of preoperative intratumoral injection of immature dendritic cells and OK-432 for resectable pancreatic cancer patients. J Hepatobiliary Pancreat Sci, 2012. 19(4): p. 465-75.
256. Zumwalt, T. J., et al., Active secretion of CXCL10 and CCL5 from colorectal cancer microenvironments associates with GranzymeB+ CD8+ T-cell infiltration. Oncotarget, 2015. 6(5): p. 2981-91.
257. Ochsenbein, A. F., Principles of tumor immunosurveillance and implications for immunotherapy. Cancer Gene Ther, 2002. 9(12): p. 1043-55.
258. Ochsenbein, A. F., et al., Roles of tumour localization, second signals and cross priming in cytotoxic T-cell induction. Nature, 2001. 411(6841): p. 1058-64.
259. Buhtoiarov, I. N., et al., CD40 ligation activates murine macrophages via an IFN-gamma-dependent mechanism resulting in tumor cell destruction in vitro. J Immunol, 2005. 174(10): p. 6013-22.
260. Egilmez, N. K., et al., Human CD4+ effector T cells mediate indirect interleukin-12-and interferon-gamma-dependent suppression of autologous HLA-negative lung tumor xenografts in severe combined immunodeficient mice. Cancer Res, 2002. 62(9): p. 2611-7.
261. Pace, J. L., et al., Recombinant mouse gamma interferon induces the priming step in macrophage activation for tumor cell killing. J Immunol, 1983. 130(5): p. 2011-3.
262. Heusinkveld, M., et al., M2 macrophages induced by prostaglandin E2 and IL-6 from cervical carcinoma are switched to activated M1 macrophages by CD4+ Th1 cells. J Immunol, 2011. 187(3): p. 1157-65.
263. Hu, G. and S. Wang, Tumor-infiltrating CD45RO+ Memory T Lymphocytes Predict Favorable Clinical Outcome in Solid Tumors. Sci Rep, 2017. 7(1): p. 10376.
264. Lohneis, P., et al., Cytotoxic tumour-infiltrating T lymphocytes influence outcome in resected pancreatic ductal adenocarcinoma. Eur J Cancer, 2017. 83: p. 290-301.
265. Liu, S., et al., Role of Cytotoxic Tumor-Infiltrating Lymphocytes in Predicting Outcomes in Metastatic HER2-Positive Breast Cancer: A Secondary Analysis of a Randomized Clinical Trial. JAMA Oncol, 2017: p. e172085.
266. Berntsson, J., et al., The clinical impact of tumour-infiltrating lymphocytes in colorectal cancer differs by anatomical subsite: A cohort study. Int J Cancer, 2017. 141(8): p. 1654-1666.
267. Xu, Y., et al., Higher Numbers of T-Bet+ Tumor-Infiltrating Lymphocytes Associate with Better Survival in Human Epithelial Ovarian Cancer. Cell Physiol Biochem, 2017. 41(2): p. 475-483.
268. Melief, S. M., et al., Long-term Survival and Clinical Benefit from Adoptive T-cell Transfer in Stage IV Melanoma Patients Is Determined by a Four-Parameter Tumor Immune Signature. Cancer Immunol Res, 2017. 5(2): p. 170-179.
269. Scurr, M. J., et al., Low-dose cyclophosphamide induces anti-tumor T-cell responses which associate with survival in metastatic colorectal cancer. Clin Cancer Res, 2017.
270. Wang, L., et al., Arsenic trioxide is an immune adjuvant in liver cancer treatment. Mol Immunol, 2017. 81: p. 118-126.
271. Ouyang, Z., et al., Regulatory T cells in the immunotherapy of melanoma. Tumour Biol, 2016. 37(1): p. 77-85.
272. Dimeloe, S., et al., Human regulatory T cells lack the cyclophosphamide-extruding transporter ABCB1 and are more susceptible to cyclophosphamide-induced apoptosis. Eur J Immunol, 2014. 44(12): p. 3614-20.
273. Camisaschi, C., et al., Effects of cyclophosphamide and IL-2 on regulatory CD4+ T cell frequency and function in melanoma patients vaccinated with HLA-class I peptides: impact on the antigen-specific T cell response. Cancer Immunol Immunother, 2013. 62(5): p. 897-908.
274. Kan, S., et al., Suppressive effects of cyclophosphamide and gemcitabine on regulatory T-cell induction in vitro. Anticancer Res, 2012. 32(12): p. 5363-9.
275. Farsam, V., et al., Antitumor and immunomodulatory properties of artemether and its ability to reduce CD4+ CD25+ FoxP3+ T reg cells in vivo. Int Immunopharmacol, 2011. 11(11): p. 1802-8.
276. Lawrence, H. S. and A. M. Pappenheimer, Jr., Transfer of delayed hypersensitivity to diphtheria toxin in man. J Exp Med, 1956. 104(3): p. 321-35.
277. Rosenfeld, S. and D. Dressler, Transfer factor: a subcellular component that transmits information for specific immune responses. Proc Natl Acad Sci USA, 1974. 71(6): p. 2473-7.
278. Dressler, D. and S. Rosenfeld, On the chemical nature of transfer factor. Proc Natl Acad Sci USA, 1974. 71(11): p. 4429-34.
279. Shifrine, M. and R. Scibienski, Transfer factor—hypotheses for its structure and function. Oncology, 1975. 32(5-6): p. 269-74.
280. Kirkpatrick, C. H., Properties and activities of transfer factor. J Allergy Clin Immunol, 1975. 55(6): p. 411-21.
281. Burger, D. R., et al., Human transfer factor: fractionation and biologic activity. J Immunol, 1976. 117(3): p. 789-96.
282. Berron-Perez, R., et al., Indications, usage, and dosage of the transfer factor. Rev Alerg Mex, 2007. 54(4): p. 134-9.
283. Alexandrescu, D. T., C. A. Dasanu, and C. L. Kauffman, Acute scurvy during treatment with interleukin-2. Clin Exp Dermatol, 2009. 34(7): p. 811-4.
284. Anthony, H. M. and C. J. Schorah, Severe hypovitaminosis C in lung-cancer patients: the utilization of vitamin C in surgical repair and lymphocyte-related host resistance. Br J Cancer, 1982. 46(3): p. 354-67.
285. McMurray, D. N., Cell-mediated immunity in nutritional deficiency. Prog Food Nutr Sci, 1984. 8(3-4): p. 193-228.
286. http://www.highbeam.com/doc/1G1-186526887.html.
287. Marcus, S. L., et al., Severe hypovitaminosis C occurring as the result of adoptive immunotherapy with high-dose interleukin 2 and lymphokine-activated killer cells. Cancer Res, 1987. 47(15): p. 4208-12.
288. Marcus, S. L., et al., Hypovitaminosis C in patients treated with high-dose interleukin 2 and lymphokine-activated killer cells. Am J Clin Nutr, 1991. 54(6 Suppl): p. 1292S-1297S.
289. Yeom, C. H., G. C. Jung, and K. J. Song, Changes of terminal cancer patients' health-related quality of life after high dose vitamin C administration. J Korean Med Sci, 2007. 22(1): p. 7-11.
290. Murata, A., F. Morishige, and H. Yamaguchi, Prolongation of survival times of terminal cancer patients by administration of large doses of ascorbate. Int J Vitam Nutr Res Suppl, 1982. 23: p. 103-13.
291. Cameron, E. and A. Campbell, The orthomolecular treatment of cancer. II. Clinical trial of high-dose ascorbic acid supplements in advanced human cancer. Chem Biol Interact, 1974. 9(4): p. 285-315.
292. Riordan, N. H., et al., Intravenous ascorbate as a tumor cytotoxic chemotherapeutic agent. Med Hypotheses, 1995. 44(3): p. 207-13.
293. Deubzer, B., et al., H(2)O(2)-mediated cytotoxicity of pharmacologic ascorbate concentrations to neuroblastoma cells: potential role of lactate and ferritin. Cell Physiol Biochem. 25(6): p. 767-74.
294. Gilloteaux, J., et al., Cell damage and death by autoschizis in human bladder (RT4) carcinoma cells resulting from treatment with ascorbate and menadione. Ultrastruct Pathol. 34(3): p. 140-60.
295. Cullen, J. J., Ascorbate induces autophagy in pancreatic cancer. Autophagy. 6(3): p. 421-2.
296. Takemura, Y., et al., High dose of ascorbic acid induces cell death in mesothelioma cells. Biochem Biophys Res Commun. 394(2): p. 249-53.
297. Verrax, J., et al., In situ modulation of oxidative stress: a novel and efficient strategy to kill cancer cells. Curr Med Chem, 2009. 16(15): p. 1821-30.
298. Fromberg, A., et al., Ascorbate exerts anti-proliferative effects through cell cycle inhibition and sensitizes tumor cells towards cytostatic drugs. Cancer Chemother Pharmacol.
299. Pollard, H. B., et al., Pharmacological ascorbic acid suppresses syngeneic tumor growth and metastases in hormone-refractory prostate cancer. In Vivo. 24(3): p. 249-55.
300. Chen, Q., et al., Pharmacologic doses of ascorbate act as a prooxidant and decrease growth of aggressive tumor xenografts in mice. Proc Natl Acad Sci USA, 2008. 105(32): p. 11105-9.
301. Padayatty, S. J., et al., Intravenously administered vitamin C as cancer therapy: three cases. CMAJ, 2006. 174(7): p. 937-42.
302. Mikirova, N. A., T. E. Ichim, and N. H. Riordan, Anti-angiogenic effect of high doses of ascorbic acid. J Transl Med, 2008. 6: p. 50.
303. Ashino, H., et al., Novel function of ascorbic acid as an angiostatic factor. Angiogenesis, 2003. 6(4): p. 259-69.
304. Mikirova, N. A., J. J. Casciari, and N. H. Riordan, Ascorbate inhibition of angiogenesis in aortic rings ex vivo and subcutaneous Matrigel plugs in vivo. J Angiogenes Res. 2: p. 2.
305. Yeom, C. H., et al., High dose concentration administration of ascorbic acid inhibits tumor growth in BALB/C mice implanted with sarcoma 180 cancer cells via the restriction of angiogenesis. J Transl Med, 2009. 7: p. 70.
306. Muellner, M. K., et al., Vitamin C inhibits NO-induced stabilization of HIF-1alpha in HUVECs. Free Radic Res. 44(7): p. 783-91.
307. Horak, P., et al., Negative feedback control of HIF-1 through REDD1-regulated ROS suppresses tumorigenesis. Proc Natl Acad Sci USA. 107(10): p. 4675-80.
308. Gao, P., et al., HIF-dependent antitumorigenic effect of antioxidants in vivo. Cancer Cell, 2007. 12(3): p. 230-8.
309. Kuiper, C., et al., Low ascorbate levels are associated with increased hypoxia-inducible factor-1 activity and an aggressive tumor phenotype in endometrial cancer. Cancer Res. 70(14): p. 5749-58.
310. http://www.clinicaltrials.gov/ct2/show/NCT00441207.
311. http://www.clinicaltrials.gov/ct2/show/NCT01080352.
312. http://www.clinicaltrials.gov/ct2/show/NCT00626444.
313. http://www.clinicaltrials.gov/ct2/show/NCT01125449.
314. http://www.clinicaltrials.gov/ct2/show/NCT01050621.
315. http://www.clinicaltrials.gov/ct2/show/NCT00954525.
316. Tatla, S., et al., The role of reactive oxygen species in triggering proliferation and IL-2 secretion in T cells. Free Radic Biol Med, 1999. 26(1-2): p. 14-24.
317. Williams, M. S. and J. Kwon, T cell receptor stimulation, reactive oxygen species, and cell signaling. Free Radic Biol Med, 2004. 37(8): p. 1144-51.
318. Schwager, J. and J. Schulze, Influence of ascorbic acid on the response to mitogens and interleukin production of porcine lymphocytes. Int J Vitam Nutr Res, 1997. 67(1): p. 10-6.
319. Eylar, E., et al., Sustained levels of ascorbic acid are toxic and immunosuppressive for human T cells. P R Health Sci J, 1996. 15(1): p. 21-6.
320. Huwyler, T., A. Hirt, and A. Morell, Effect of ascorbic acid on human natural killer cells. Immunol Lett, 1985. 10(3-4): p. 173-6.
321. Tan, P. H., et al., Inhibition of NF-kappa B and oxidative pathways in human dendritic cells by antioxidative vitamins generates regulatory T cells. J Immunol, 2005. 174(12): p. 7633-44.
322. Chen, S., L. Yang, and Y. Li, TCR zeta chain expression in T cells from patients with CML. Hematology, 2009. 14(2): p. 95-100.
323. Kulkarni, D. P., et al., Mechanisms involved in the down-regulation of TCR zeta chain in tumor versus peripheral blood of oral cancer patients. Int J Cancer, 2009. 124(7): p. 1605-13.
324. Gruber, I. V., et al., Down-regulation of CD28, TCR-zeta (zeta) and up-regulation of FAS in peripheral cytotoxic T-cells of primary breast cancer patients. Anticancer Res, 2008. 28(2A): p. 779-84.
325. Pignataro, L., et al., Down-regulation of zeta chain and zeta-associated protein 70 (Zap 70) expression in circulating T lymphocytes in laryngeal squamous cell carcinoma. Anal Quant Cytol Histol, 2007. 29(1): p. 57-62.
326. Zehbe, I., et al., Different T-cell receptor (TCR) zeta chain expression in cervical cancer and its precursor lesions. Zentralbl Gynakol, 2006. 128(5): p. 266-70.
327. Ciszak, L., et al., Alterations in the expression of signal-transducing CD3 zeta chain in T cells from patients with chronic inflammatory/autoimmune diseases. Arch Immunol Ther Exp (Warsz), 2007. 55(6): p. 373-86.
328. Baniyash, M., TCR zeta-chain downregulation: curtailing an excessive inflammatory immune response. Nat Rev Immunol, 2004. 4(9): p. 675-87.
329. Pitcher, L. A. and N. S. van Oers, T-cell receptor signal transmission: who gives an ITAM? Trends Immunol, 2003. 24(10): p. 554-60.
330. Gastman, B. R., et al., Tumor-induced apoptosis of T lymphocytes: elucidation of intracellular apoptotic events. Blood, 2000. 95(6): p. 2015-23.
331. Boussiotis, V. A., et al., Differential association of protein tyrosine kinases with the T cell receptor is linked to the induction of anergy and its prevention by B7 family-mediated costimulation. J Exp Med, 1996. 184(2): p. 365-76.
332. Kim, C. W., et al., Alteration of signal-transducing molecules and phenotypical characteristics in peripheral blood lymphocytes from gastric carcinoma patients. Pathobiology, 1999. 67(3): p. 123-8.
333. Reichert, T. E., et al., Absent or low expression of the zeta chain in T cells at the tumor site correlates with poor survival in patients with oral carcinoma. Cancer Res, 1998. 58(23): p. 5344-7.
334. Zea, A. H., et al., Alterations in T cell receptor and signal transduction molecules in melanoma patients. Clin Cancer Res, 1995. 1(11): p. 1327-35.
335. Healy, C. G., et al., Impaired expression and function of signal-transducing zeta chains in peripheral T cells and natural killer cells in patients with prostate cancer. Cytometry, 1998. 32(2): p. 109-19.
336. Mulder, W. M., et al., T cell receptor-zeta and granzyme B expression in mononuclear cell infiltrates in normal colon mucosa and colon carcinoma. Gut, 1997. 40(1): p. 113-9.
337. Muller, D., et al., [The expression of zeta-chain of the T cell receptor as prognostic marker for patients with head and neck cancer]. Laryngorhinootologie, 2002. 81(7): p. 516-20.
338. Whiteside, T. L., Down-regulation of zeta-chain expression in T cells: a biomarker of prognosis in cancer? Cancer Immunol Immunother, 2004. 53(10): p. 865-78.
339. Eleftheriadis, T., et al., Decreased CD3+CD16+ natural killer-like T-cell percentage and zeta-chain expression accompany chronic inflammation in haemodialysis patients. Nephrology (Carlton), 2009. 14(5): p. 471-5.
340. Eleftheriadis, T., et al., Chronic inflammation and CD16+ natural killer cell zeta-chain downregulation in hemodialysis patients. Blood Purif, 2008. 26(4): p. 317-21.
341. Nambiar, M. P., et al., TCR zeta-chain abnormalities in human systemic lupus erythematosus. Methods Mol Med, 2004. 102: p. 49-72.
342. Takeuchi, T., et al., T cell abnormalities in systemic lupus erythematosus. Autoimmunity, 2005. 38(5): p. 339-46.
343. Berg, L., et al., Down-regulation of the T cell receptor CD3 zeta chain in rheumatoid arthritis (RA) and its influence on T cell responsiveness. Clin Exp Immunol, 2000. 120(1): p. 174-82.
344. Maurice, M. M., et al., Defective TCR-mediated signaling in synovial T cells in rheumatoid arthritis. J Immunol, 1997. 159(6): p. 2973-8.
345. Ammirati, E., et al., Expansion of T-cell receptor zeta dim effector T cells in acute coronary syndromes. Arterioscler Thromb Vasc Biol, 2008. 28(12): p. 2305-11.
346. Sikora, J., et al., The role of monocytes/macrophages in TCR-zeta chain downregulation and apoptosis of T lymphocytes in malignant pleural effusions. J Biol Regul Homeost Agents, 2004. 18(1): p. 26-32.
347. Markiewski, M. M., et al., Modulation of the antitumor immune response by complement. Nat Immunol, 2008. 9(11): p. 1225-35.
348. Corzo, C. A., et al., Mechanism regulating reactive oxygen species in tumor-induced myeloid-derived suppressor cells. J Immunol, 2009. 182(9): p. 5693-701.
349. Choi, J. Y., J. A. Oughton, and N. I. Kerkvliet, Functional alterations in CD11b(+)Gr-1(+) cells in mice injected with allogeneic tumor cells and treated with 2,3,7,8-tetrachlorodibenzo-p-dioxin. Int Immunopharmacol, 2003. 3(4): p. 553-70.
350. Makarenkova, V. P., et al., CD11b+/Gr-1+ myeloid suppressor cells cause T cell dysfunction after traumatic stress. J Immunol, 2006. 176(4): p. 2085-94.
351. Ezernitchi, A. V., et al., TCR zeta down-regulation under chronic inflammation is mediated by myeloid suppressor cells differentially distributed between various lymphatic organs. J Immunol, 2006. 177(7): p. 4763-72.
352. Schmielau, J. and O. J. Finn, Activated granulocytes and granulocyte-derived hydrogen peroxide are the underlying mechanism of suppression of t-cell function in advanced cancer patients. Cancer Res, 2001. 61(12): p. 4756-60.
353. Nambiar, M. P., et al., Oxidative stress is involved in the heat stress-induced downregulation of TCR zeta chain expression and TCR/CD3-mediated [Ca(2+)](i) response in human T-lymphocytes. Cell Immunol, 2002. 215(2): p. 151-61.
354. Cao, Y., et al., Forty-year journey of angiogenesis translational research. Sci Transl Med, 2011. 3(114): p. 114rv3.
355. Abdollahi, A. and J. Folkman, Evading tumor evasion: current concepts and perspectives of anti-angiogenic cancer therapy. Drug Resist Updat, 2010. 13(1-2): p. 16-28.
356. Chen, H. X., R. E. Gore-Langton, and B. D. Cheson, Clinical trials referral resource: Current clinical trials of the anti-VEGF monoclonal antibody bevacizumab. Oncology (Williston Park), 2001. 15(8): p. 1017, 1020, 1023-6.
357. Krupitskaya, Y. and H. A. Wakelee, Ramucirumab, a fully human mAb to the transmembrane signaling tyrosine kinase VEGFR-2 for the potential treatment of cancer. Curr Opin Investig Drugs, 2009. 10(6): p. 597-605.
358. Abrams, T. J., et al., SU11248 inhibits KIT and platelet-derived growthfactor receptor beta in preclinical models of human small cell lung cancer. Mol Cancer Ther, 2003. 2(5): p. 471-8.
359. Carlisle, B., et al., Benefit, Risk, and Outcomes in Drug Development: A Systematic Review of Sunitinib. J Natl Cancer Inst, 2016. 108(1).
360. Izzedine, H., et al., Sunitinib malate. Cancer Chemother Pharmacol, 2007. 60(3): p. 357-64.
361. Viola, D., V. Cappagli, and R. Elisei, Cabozantinib (XLI84) for the treatment of locally advanced or metastatic progressive medullary thyroid cancer. Future Oncol, 2013. 9(8): p. 1083-92.
362. Roy, S., et al., A novel multiple tyrosine-kinase targeted agent to explore the future perspectives of anti-angiogenic therapy for the treatment of multiple solid tumors: cabozantinib. Anticancer Agents Med Chem, 2015. 15(1): p. 37-47.
363. Tannir, N. M., G. Schwab, and V. Grunwald, Cabozantinib: an Active Novel Multikinase Inhibitor in Renal Cell Carcinoma. Curr Oncol Rep, 2017. 19(2): p. 14.
364. Yakes, F. M., et al., Cabozantinib (XLI84), a novel MET and VEGFR2 inhibitor, simultaneously suppresses metastasis, angiogenesis, and tumor growth. Mol Cancer Ther, 2011. 10(12): p. 2298-308.
365. Gril, B., et al., The B-Raf status of tumor cells may be a significant determinant of both antitumor and anti-angiogenic effects of pazopanib in xenograft tumor models. PLoS One, 2011. 6(10): p. e25625.
366. van Geel, R. M., J. H. Beijnen, and J. H. Schellens, Concise drug review: pazopanib and axitinib. Oncologist, 2012. 17(8): p. 1081-9.
367. Ferrero, S., et al., Pharmacokinetic drug evaluation of pazopanib for the treatment of uterine leiomyosarcomas. Expert Opin Drug Metab Toxicol, 2017. 13(8): p. 881-889.
368. Gaumann, A. K., et al., Receptor tyrosine kinase inhibitors: Are they real tumor killers?Int J Cancer, 2016. 138(3): p. 540-54.
369. Miura, K., et al., The preclinical development of regorafenib for the treatment of colorectal cancer. Expert Opin Drug Discov, 2014. 9(9): p. 1087-101.
370. Rimassa, L., et al., Regorafenib for the treatment of unresectable hepatocellular carcinoma. Expert Rev Anticancer Ther, 2017. 17(7): p. 567-576.
371. Ohga, N., et al., Heterogeneity of tumor endothelial cells: comparison between tumor endothelial cells isolated from high-and low-metastatic tumors. Am J Pathol, 2012. 180(3): p. 1294-307.
372. Akiyama, K., et al., Tumor endothelial cells acquire drug resistance by MDR1 up-regulation via VEGF signaling in tumor microenvironment. Am J Pathol, 2012. 180(3): p. 1283-93.
373. Ohmura-Kakutani, H., et al., Identification of tumor endothelial cells with high aldehyde dehydrogenase activity and a highly angiogenic phenotype. PLoS One, 2014. 9(12): p. e113910.
374. Sruthi, T. V., et al., Horizontal transfer of miR-23a from hypoxic tumor cell colonies can induce angiogenesis. J Cell Physiol, 2017.
375. Kosaka, N., et al., Neutral sphingomyelinase 2 (nSMase2)-dependent exosomal transfer of angiogenic microRNAs regulate cancer cell metastasis. J Biol Chem, 2013. 288(15): p. 10849-59.
376. Zhang, Y., et al., Tumacrophage: macrophages transformed into tumor stem-like cells by virulent genetic material from tumor cells. Oncotarget, 2017. 8(47): p. 82326-82343.
377. Abdouh, M., et al., Exosomes isolated from cancer patients' sera transfer malignant traits and confer the same phenotype of primary tumors to oncosuppressor-mutated cells. J Exp Clin Cancer Res, 2017. 36(1): p. 113.
378. Semina, S. E., et al., Horizontal Transfer of Tamoxifen Resistance in MCF-7 Cell Derivates: Proteome Study. Cancer Invest, 2017. 35(8): p. 506-518.
379. Cai, J., et al., Functional transferred DNA within extracellular vesicles. Exp Cell Res, 2016. 349(1): p. 179-183.
380. Hamam, D., et al., Transfer of malignant trait to BRCA1 deficient human fibroblasts following exposure to serum of cancer patients. J Exp Clin Cancer Res, 2016. 35: p. 80.
381. Berridge, M. V., L. Dong, and J. Neuzil, Mitochondrial DNA in Tumor Initiation, Progression, and Metastasis: Role of Horizontal mtDNA Transfer. Cancer Res, 2015. 75(16): p. 3203-8.
382. Chen, W. X., et al., Exosomes from drug-resistant breast cancer cells transmit chemoresistance by a horizontal transfer of microRNAs. PLoS One, 2014. 9(4): p. e95240.
383. Zhu, X., et al., BCR-ABL1-positive microvesicles transform normal hematopoietic transplants through genomic instability: implications for donor cell leukemia. Leukemia, 2014. 28(8): p. 1666-75.
384. Stein, J., et al., Origin of leukemic relapse after bone marrow transplantation: comparison of cytogenetic and molecular analyses. Blood, 1989. 73(7): p. 2033-40.
385. Angara, K., T. F. Borin, and A. S. Arbab, Vascular Mimicry: A Novel Neovascularization Mechanism Driving Anti-Angiogenic Therapy (AAT) Resistance in Glioblastoma. Transl Oncol, 2017. 10(4): p. 650-660.
386. Sood, A. K., et al., The clinical significance of tumor cell-lined vasculature in ovarian carcinoma: implications for anti-vasculogenic therapy. Cancer Biol Ther, 2002. 1(6): p. 661-4.
387. Mahase, S., et al., Hypoxia-Mediated Mechanisms Associated with Antiangiogenic Treatment Resistance in Glioblastomas. Am J Pathol, 2017. 187(5): p. 940-953.
388. Pinto, M. P., et al., Escaping Antiangiogenic Therapy: Strategies Employed by Cancer Cells. Int J Mol Sci, 2016. 17(9).
389. Schnegg, C. I., et al., Induction of Vasculogenic Mimicry Overrides VEGF-A Silencing and Enriches Stem-like Cancer Cells in Melanoma. Cancer Res, 2015. 75(8): p. 1682-90.
390. Soda, Y., et al., Transdifferentiation of glioblastoma cells into vascular endothelial cells. Proc Natl Acad Sci USA, 2011. 108(11): p. 4274-80.
391. Li, Q., et al., Angiogenesis inhibitors for the treatment of small cell lung cancer (SCLC): A meta-analysis of 7 randomized controlled trials. Medicine (Baltimore), 2017. 96(13): p. e6412.
392. Lombardi, G., et al., Effectiveness of antiangiogenic drugs in glioblastoma patients: A systematic review and meta-analysis of randomized clinical trials. Crit Rev Oncol Hematol, 2017. 111: p. 94-102.
393. Roviello, G., et al., The role of bevacizumab in solid tumours: A literature based meta-analysis of randomised trials. Eur J Cancer, 2017. 75: p. 245-258.
394. Shih, T. and C. Lindley, Bevacizumab: an angiogenesis inhibitor for the treatment of solid malignancies. Clin Ther, 2006. 28(11): p. 1779-802.
395. Chase, J. L., Clinical use of anti-vascular endothelial growth factor monoclonal antibodies in metastatic colorectal cancer. Pharmacotherapy, 2008. 28(11 Pt 2): p. 23S-30S.
396. Yu, J., et al., Efficacy and safety of angiogenesis inhibitors in advanced gastric cancer: a systematic review and meta-analysis. J Hematol Oncol, 2016. 9(1): p. 111.
397. Ciliberto, D., et al., A systematic review and meta-analysis of randomized trials on the role of targeted therapy in the management of advanced gastric cancer: Evidence does not translate? Cancer Biol Ther, 2015. 16(8): p. 1148-59.
398. Welsh, S. J. and K. Fife, Pazopanib for the treatment of renal cell carcinoma. Future Oncol, 2015. 11(8): p. 1169-79.
399. Khan, K., D. Cunningham, and I. Chau, Targeting Angiogenic Pathways in Colorectal Cancer: Complexities, Challenges and Future Directions. Curr Drug Targets, 2017. 18(1): p. 56-71.
400. Iacovelli, R., et al., Inhibition of the VEGF/VEGFR pathway improves survival in advanced kidney cancer: a systematic review and meta-analysis. Curr Drug Targets, 2015. 16(2): p. 164-70.
401. Piperdi, B., A. Merla, and R. Perez-Soler, Targeting angiogenesis in squamous non-small cell lung cancer. Drugs, 2014. 74(4): p. 403-13.
402. von Baumgarten, L., et al., Bevacizumab has differential and dose-dependent effects on glioma blood vessels and tumor cells. Clin Cancer Res, 2011. 17(19): p. 6192-205.
403. Yuan, F., et al., Time-dependent vascular regression and permeability changes in established human tumor xenografts induced by an anti-vascular endothelial growth factor/vascular permeability factor antibody. Proc Natl Acad Sci USA, 1996. 93(25): p. 14765-70.
404. Aalders, K. C., et al., Anti-angiogenic treatment in breast cancer: Facts, successes, failures and future perspectives. Cancer Treat Rev, 2017. 53: p. 98-110.
405. Wang, M., et al., Rapamycin suppresses angiogenesis and lymphangiogenesis in melanoma by downregulating VEGF-A/VEGFR-2 and VEGF-C/VEGFR-3 expression. Onco Targets Ther, 2019. 12: p. 4643-4654.

1. A method of treating cancer comprising: a) examining a cancer patient for tumor and host associated abnormalities; b) addressing said abnormalities; c) providing one or more therapeutic interventions; and d) providing immunological support to avoid tumor recurrence.

2. The method of claim 1, wherein said therapeutic capable of reducing tumor-associated immune suppression is an antioxidant selected from the group comprising of: a) n-acetylcysteine; b) intravenous ascorbic acid; c) pterostilbene; d) vitamin k3; e) resveratrol; f) alpha lipoic acid; g) quercetin; h) kaempferol; i) myricetin; j) apigenin; k) luteolin; l) curcumin; and m) caffeic acid.

3. The method of claim 1, wherein said therapeutic capable of reducing tumor-associated immune suppression is a phosphodiesterase (PDE)-5 inhibitor, wherein said PDE-5 inhibitor is selected from a group comprising of: a) Acetildenafi; b) Aildenafil; c) Avanafil; d) Benzamidenafil; e) Homosildenafil; f) Icariin; g) Lodenafil; h) Mirodenafil; i) Nitrosoprodenafil; j) Sildenafil; k) Sulfoaildenafil; l) Tadalafil; m) Udenafil; n) Vardenafil; and o) Zaprinast.

4. The method of claim 1, wherein said therapeutic capable of reducing tumor-associated immune suppression is an agent capable of reducing VEGF, wherein said agent capable of reducing said VEGF is selected from a group comprising of: a) Avastin; b) Ciclopirox; c) penicillamine; d) tetrathiomolybdate; e) fish oil; f) selenium; g) green tea polyphenols; h) glycine; i) zinc; j) cirsimaritin; k) Eupafolin; l) Andrographolide; m) Procyanidin B2; n) Procyanidin B3; o) 6-O-angeloylenolin; p) Cyperenoic acid; q) Penduliflaworosin; r) Tylophorine; s) Ellagic acid; t) brucine; u) Punarnavine; v) Raddeanin A; w) Platycodin D; x) withanone; y) 4-Hydroxyphenylacetic acid; z) trans-ethyl p-methoxycinnamate; aa) Decursin; ab) decursinol angelate; and ac) Artesunate.

5. The method of claim 1, wherein said therapeutic capable of reducing tumor-associated immune suppression is a checkpoint inhibitor, wherein said checkpoint inhibitor is an agent capable of blocking molecules selected from a group comprising of: a) PD-1; b) PD-Li; c) CTLA-4; d) LAG-3; e) TIGIT; f) KIR; g) indolamine 2,3 deoxygenase; h) NR2F6; i) TIM-3; j) ILT-3; and k) GITR.

6. The method of claim 1, wherein said patient is immunized with a tumor antigen, wherein said tumor antigen possesses similarity to said tumor which said patient is afflicted by.

7. The method of claim 6, wherein said tumor antigen is derived from a histologically similar tumor to which said patient is afflicted with.

8. The method of claim 7, wherein said tumor antigen is derived by lysis of histologically similar tumors.

9. The method of claim 7, wherein said tumor antigen is derived by mRNA extraction of histologically similar tumors.

10. The method of claim 7, wherein said tumor antigen is derived by exosome extraction of histologically similar tumors.

11. The method of claim 6, wherein said tumor antigen is a tumor associated antigen.

12. The method of claim 11, wherein said tumor associated antigen is selected from a group comprising of: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globol1; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak) HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

13. The method of claim 12, wherein a peptide or plurality of peptides derived from said antigens are used for immunization.

14. The method of claim 13, wherein said peptides used for immunization are matched with HLA haplotype of said patient in need of therapy.

15. The method of claim 13, wherein said peptides are altered peptide ligands.

16. The method of claim 11, wherein said immunization with said tumor antigen is performed together with an adjuvant.

17. The method of claim 16, wherein said adjuvant is a stimulator of antigen presentation.

18. The method of claim 17, wherein said stimulator of antigen presentation is a toll like receptor (TLR).

19. The method of claim 18, wherein said toll like receptor is activated by peptide possessing at least 80 percent homology to the sequence (SEQ ID NO: 1) EFDVILKAAGANKVAVIKAVRGATGLGLKEAKDLVESAPAALKEGVSKDDAEALKKAL EEAGAEVEVK.

20. The method of claim 18, wherein said TLR is activated by High mobility group box 1 (HMGB-1) protein.
